(19) United States
(12) Patent Application Publication
(10) Pub. No.: US 2010/0207517 A1
(43) Pub. Date: Aug. 19, 2010

(54) Oxadiazole Derivative, and Light-Emitting Element and Light-Emitting Device Using Oxadiazole Derivative

(75) Inventors: Hiroko NOMURA, Fukuoka (JP); Sachiko KAWAKAMI, Atsugi (JP); Nobuharu OHSAWA, Zama (JP); Satoshi SEO, Kawasaki (JP)

Correspondence Address: COOK ALEX LTD, SUITE 2850, 200 WEST ADAMS STREET, CHICAGO, IL, 60606, US

(73) Assignee: Semiconductor Energy Laboratory Co., Ltd.

(21) Appl. No.: 12/707,022

(22) Filed: Feb. 17, 2010

(30) Foreign Application Priority Data
Feb. 19, 2009 (JP) 2009-036626

(51) Int. Cl. H01J 1/63; C07D 413/10

(52) U.S. Cl. 313/504; 548/145

(57) ABSTRACT

An object is to provide a novel oxadiazole derivative that has high excitation energy, particularly high triplet excitation energy, or to provide a new oxadiazole derivative that is a bipolar substance. An oxadiazole derivative represented by General Formula (G1) is provided. In the formula, R¹ and R² independently represent a hydrogen atom, an alkyl group having 1 to 4 carbon atoms, or a substituted or unsubstituted aryl group having 6 to 10 carbon atoms. At least one of R¹ and R² represents a substituted or unsubstituted aryl group having 6 to 10 carbon atoms in a ring. Ar¹ represents a substituted or unsubstituted aryl group having 6 to 10 carbon atoms in a ring.

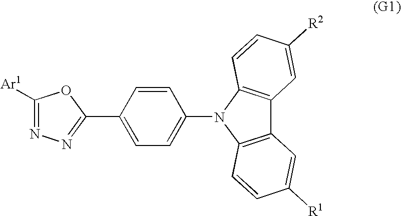

### BACKGROUND OF THE INVENTION

1. Field of the Invention

The present invention relates to an oxadiazole derivative, and a light-emitting element and a light-emitting device each using the oxadiazole derivative.

2. Description of the Related Art

In recent years, research and development of light-emitting elements using electroluminescence have been extensively conducted. In the basic structure of such a light-emitting element, a layer including a light-emitting substance is interposed between a pair of electrodes. By applying a voltage to this element, light emission can be obtained from the light-emitting substance.

Since this type of light-emitting element is a self-luminous type, it has advantages over a liquid crystal display in that visibility of a pixel is high and that no backlight is needed. Therefore, light-emitting elements are thought to be suitable as flat panel display elements. Further, such a light-emitting element also has advantages in that the element can be formed to be thin and lightweight and that response speed is very high.

Further, since this type of light-emitting element can be formed to have a film shape, surface light emission can be easily obtained. This feature is difficult to realize with point light sources typified by a filament lamp and an LED or with linear light sources typified by a fluorescent light. Therefore, such light-emitting elements also have a high utility value.

Light-emitting elements using electroluminescence are broadly classified according to whether their light-emitting substance is an organic compound or an inorganic compound. When an organic compound is used as a light-emitting substance, the emission mechanism is as follows. First, a voltage is applied to a light-emitting element. This allows electrons and holes to be injected from a pair of electrodes into a layer including a light-emitting organic compound. Accordingly, the light-emitting organic compound is raised to an excited state. Then, recombining carriers (electrons and holes) emit light in transition from the excited state to the ground state.

Because of the above mechanism, the light-emitting element is called a current-excitation light-emitting element. Note that an excited state of an organic compound can be of two types: a singlet excited state and a triplet excited state, and luminescence from the singlet excited state (S*) is referred to as fluorescence, and luminescence from the triplet excited state (T*) is referred to as phosphorescence. Furthermore, it is thought that the ratio of S* to T* in a light-emitting element is statistically 1:3.

At room temperature, a compound that converts a singlet excited state into luminescence (hereinafter referred to as a fluorescent compound) does not exhibit luminescence from a triplet excited state (phosphorescence). Therefore, the internal quantum efficiency (ratio of generated photons to injected carriers) of a light-emitting element using a fluorescent compound is thought to have a theoretical limit of 25% on the basis that S*:T*=1:3.

In contrast, by using a compound that converts a triplet excited state into luminescence (hereinafter referred to as a phosphorescent compound), an internal quantum efficiency of 75 to 100% can theoretically be achieved. That is, emission efficiency can be three to four times as high as that of a fluorescent compound. From such a reason, in order to achieve a light-emitting element with high efficiency, a light-emitting element using a phosphorescent compound has been actively developed recently (e.g., see Non Patent Document 1).

When a light-emitting layer of a light-emitting element is formed using a phosphorescent compound as described above, in order to suppress concentration quenching of the phosphorescent compound or quenching due to triplet-triplet annihilation, the light-emitting layer is often formed so that the phosphorescent compound is dispersed in a matrix including another substance. In that case, a substance serving as a matrix may be referred to as a host material, and a substance that is dispersed in a matrix, such as a phosphorescent compound, may be referred to as a guest material.

When a phosphorescent compound is used as a guest material, a host material is needed to have triplet excitation energy (an energy difference between a ground state and a triplet excited state) higher than the phosphorescent compound. It is known that CBP, which is used as a host material in Non-Patent Document 1, has higher triplet excitation energy than a phosphorescent compound which exhibits emission of green to red light, and is widely used as a host material in the phosphorescent compound.

However, although CBP has high triplet excitation energy, it is poor in ability to receive holes or electrons; therefore, there is a problem in that driving voltage gets higher. Therefore, a substance that has high triplet excitation energy and also can easily accept or transport both holes and electrons (i.e. a bipolar substance) is required as a host material for a phosphorescent compound.

Furthermore, because singlet excitation energy (difference in energy between a ground state and a singlet excited state) is greater than triplet excitation energy, a material that has high triplet excitation energy will also have high singlet excitation energy. Consequently, a substance that has high triplet excitation energy and bipolar character is also useful in a light-emitting element formed using a fluorescent compound as a light-emitting substance.

### REFERENCE

### [Non-Patent Document]

### [Non-Patent Document 1] M. A. Baldo, etc., Applied Physics Letters, vol. 75, No. 1, pp. 4-6, 1999

### SUMMARY OF THE INVENTION

In view of the foregoing, an object of one embodiment of the present invention which is disclosed in this specification and the like (including at least the specification, the scope of claims, and the drawings) is to provide a novel oxadiazole derivative as a substance having high excitation energy, particularly as a substance having high triplet excitation energy. Another object is to provide a novel oxadiazole derivative that is a bipolar substance. Yet another object is to improve element characteristics of a light-emitting element. Still another object is to provide a light-emitting device and an electronic device each having low power consumption and long lifetime.

One embodiment of the disclosed invention is an oxadiazole derivative represented by the following General Formula (G1).

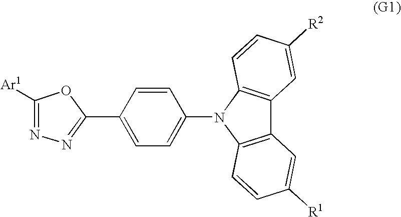

In General Formula (G1), R¹ and R² independently represent a hydrogen atom, an alkyl group having 1 to 4 carbon atoms, or a substituted or unsubstituted aryl group having 6 to 10 carbon atoms. Note that at least one of R¹ and R² represents a substituted or unsubstituted aryl group having 6 to 10 carbon atoms in a ring. In addition, Ar¹ represents a substituted or unsubstituted aryl group having 6 to 10 carbon atoms in a ring. In any of R¹, R², and Ar¹, a substituent is an alkyl group having 1 to 4 carbon atoms or an aryl group having 6 to 10 carbon atoms in a ring.

Another embodiment of the disclosed invention is an oxadiazole derivative represented by the following General Formula (G2).

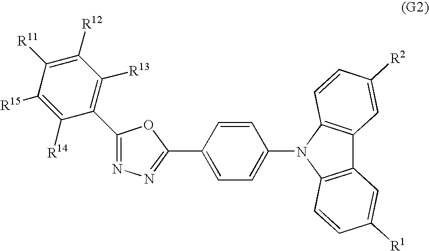

In General Formula (G1), R¹ and R² independently represent a hydrogen atom, an alkyl group having 1 to 4 carbon atoms, or a substituted or unsubstituted aryl group having 6 to 10 carbon atoms. Note that at least one of R¹ and R² represents a substituted or unsubstituted aryl group having 6 to 10 carbon atoms in a ring. In R¹ and R², a substituent is an alkyl group having 1 to 4 carbon atoms or an aryl group having 6 to 10 carbon atoms in a ring. In addition, R¹¹ to R¹⁵ independently represent a hydrogen atom, an alkyl group having 1 to 4 carbon atoms, or an aryl group having 6 to 10 carbon atoms in a ring.

Another embodiment of the disclosed invention is an oxadiazole derivative represented by the following General Formula (G3).

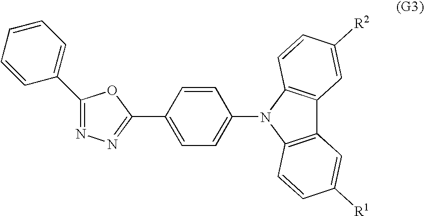

In the formula (G3), R¹ and R² independently represent a hydrogen atom, an alkyl group having 1 to 4 carbon atoms, or a substituted or unsubstituted aryl group having 6 to 10 carbon atoms. Note that at least one of R¹ and R² represents a substituted or unsubstituted aryl group having 6 to 10 carbon atoms in a ring. In R¹ and R², a substituent is an alkyl group having 1 to 4 carbon atoms or an aryl group having 6 to 10 carbon atoms in a ring.

Still another embodiment of the disclosed invention is an oxadiazole derivative represented by the following Structural Formula (G4).

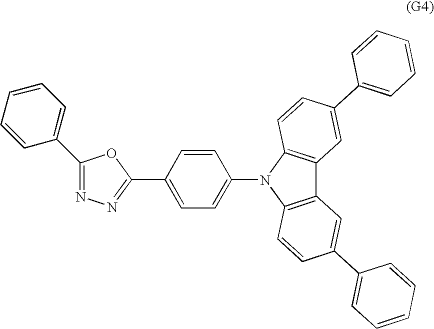

Yet another embodiment of the disclosed invention is an oxadiazole derivative represented by the following Structural Formula (G5).

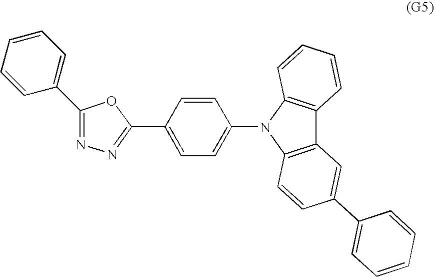

The above oxadiazole derivatives can be used as either a host material or a guest material in a light-emitting layer. Thus, a still further embodiment of the disclosed invention is a light-emitting element that has a light-emitting layer including any of the above-described oxadiazole derivatives.

Further, the above oxadiazole derivatives are suitable for use as a host material in a light-emitting layer. Thus, another embodiment of the disclosed invention is a light-emitting element that has a light-emitting layer including any of the above-described oxadiazole derivatives and a light-emitting substance.

Furthermore, since the above oxadiazole derivatives have high triplet excitation energy, they are preferably used as a host material in a light-emitting layer that includes a phosphorescent compound as a light-emitting substance. Thus, still another embodiment of the disclosed invention is a light-emitting element in which the above light-emitting substance is a phosphorescent compound.

Moreover, since the above oxadiazole derivatives have high excitation energy, a layer including any of these oxadiazole derivatives is preferably provided so as to be in contact with a light-emitting layer. Thus, still another embodiment of the disclosed invention is a light-emitting element in which the layer including any of the above-described oxadiazole derivatives is provided in contact with a light-emitting layer. Such a structure can prevent excitons generated in the light-emitting layer from diffusing out into another layer. This leads to a light-emitting element having high emission efficiency.

Furthermore, another embodiment of the disclosed invention is a light-emitting device formed using any of the above-described light-emitting elements and an electronic device formed using the light-emitting device.

Note that the term light-emitting device in this specification and the like includes an image display device, a light-emitting device, a light source (including a lighting device), and the like. Further, the category of the light-emitting device includes: a module in which a connector such as a flexible printed circuit (FPC), a tape automated bonding (TAB) tape, a tape carrier package (TCP), or the like is attached to a light-emitting device, a module in which the top of a TAB tape, a TCP, or the like is provided with a printed wire board, a module in which an integrated circuit (IC) is directly mounted on a light-emitting element by a chip on glass (COG) technique, and the like.

According to embodiments of the disclosed invention, an oxadiazole derivative having high triplet excitation energy can be provided. Alternatively, an oxadiazole derivative that is a bipolar substance can be provided. Further alternatively, element characteristics of a light-emitting element can be improved. Still alternatively, a light-emitting device or an electronic device having low power consumption and long lifetime can be provided.

### BRIEF DESCRIPTION OF THE DRAWINGS

FIG. 1 illustrates a light-emitting element.

FIG. 2 illustrates a light-emitting element.

FIG. 3 illustrates a light-emitting element.

FIGS. 4A to 4D illustrate a passive-matrix light-emitting device.

FIG. 5 illustrates a passive-matrix light-emitting device.

FIGS. 6A and 6B illustrate an active matrix light-emitting device.

FIGS. 7A to 7E each illustrate an electronic device.

FIG. 8 illustrates lighting devices.

FIG. 9 illustrates a light-emitting element.

FIGS. 10A and 10B show ¹H NMR charts of CO11II.

FIG. 11 shows an ultraviolet-visible absorption spectrum and an emission spectrum of CO11II.

FIG. 12 shows an ultraviolet-visible absorption spectrum and an emission spectrum of CO11II.

FIG. 13 shows CV measurement results of oxidation characteristics of CO11II

FIG. 14 shows CV measurement results of reduction characteristics of CO11II.

FIGS. 15A and 15B show ¹H NMR charts of CO11III.

FIG. 16 shows an ultraviolet-visible absorption spectrum and an emission spectrum of CO11III.

FIG. 17 shows an ultraviolet-visible absorption spectrum and an emission spectrum of CO11III.

FIG. 18 shows CV measurement results of oxidation characteristics of CO11III.

FIG. 19 shows CV measurement results of reduction characteristics of CO11III.

FIG. 20 shows current density vs. luminance characteristics of Light-emitting Elements 0 to 2.

FIG. 21 shows voltage vs. luminance characteristics of Light-emitting Elements 0 to 2.

FIG. 22 shows luminance vs. current efficiency characteristics of Light-emitting Elements 0 to 2.

FIG. 23 shows emission spectra of Light-emitting Elements 0 to 2.

FIG. 24 shows time vs. normalized luminance characteristics of Light-emitting Elements 0 to 2.

FIG. 25 shows current density vs. luminance characteristics of Light-Emitting Element 3.

FIG. 26 shows voltage vs. luminance characteristics of Light-Emitting Element 3.

FIG. 27 shows luminance vs. current efficiency characteristics of Light-Emitting Element 3.

FIG. 28 shows an emission spectrum of the light-emitting element 3.

FIG. 29 shows time vs. normalized luminance characteristics of Light-emitting Element 3.

FIGS. 30A and 30B show the highest occupied molecular orbital (HOMO) and lowest unoccupied molecular orbital (LUMO) of CO11II, respectively.

FIGS. 31A and 31B show the highest occupied molecular orbital (HOMO) and lowest unoccupied molecular orbital (LUMO) of CO11III, respectively.

### DETAILED DESCRIPTION OF THE INVENTION

Hereinafter, embodiments of the present invention will be described in detail with reference to the accompanying drawings. Note that the present invention is not limited to the description of the embodiments, and it is apparent to those skilled in the art that modes and details can be modified in various ways without departing from the spirit of the present invention disclosed in this specification and the like. In addition, the structures described in various embodiments can be implemented in appropriate combination. Note that in the structure of the present invention which will be described below, identical components or components having similar functions are denoted by the same reference numerals and do not require repeated explanations.

### Embodiment 1

In Embodiment 1, the oxadiazole derivatives which are one embodiment of the disclosed invention will be described.

An oxadiazole derivative which is one embodiment of the disclosed invention is represented by General Formula (G1).

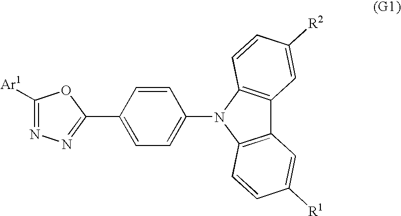

In General Formula (G1), R¹ and R² independently represent a hydrogen atom, an alkyl group having 1 to 4 carbon atoms, or a substituted or unsubstituted aryl group having 6 to 10 carbon atoms. Note that at least one of R¹ and R² represents a substituted or unsubstituted aryl group having 6 to 10 carbon atoms in a ring. In addition, Ar¹ represents a substituted or unsubstituted aryl group having 6 to 10 carbon atoms in a ring. In R¹, R², and Ar¹, a substituent is an alkyl group having 1 to 4 carbon atoms or an aryl group having 6 to 10 carbon atoms in a ring.

An oxadiazole derivative which is another embodiment of the disclosed invention is represented by General Formula (G2).

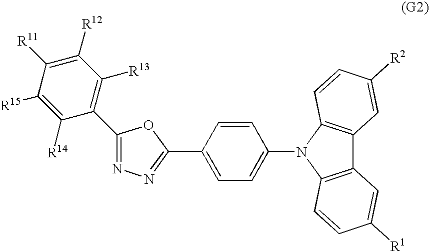

In General Formula (G2), R¹ and R² independently represent a hydrogen atom, an alkyl group having 1 to 4 carbon atoms, or a substituted or unsubstituted aryl group having 6 to 10 carbon atoms. Note that at least one of R¹ and R² represents a substituted or unsubstituted aryl group having 6 to 10 carbon atoms in a ring. In R¹ and R², a substituent is an alkyl group having 1 to 4 carbon atoms or an aryl group having 6 to 10 carbon atoms in a ring. In addition, R¹¹ to R¹⁵ independently represent a hydrogen atom, an alkyl group having 1 to 4 carbon atoms, or an aryl group having 6 to 10 carbon atoms in a ring.

As specific structures of R¹ and R² in General Formula (G1) and General Formula (G2), there are substituents represented by Structural Formulae (I-1) to (1-26).

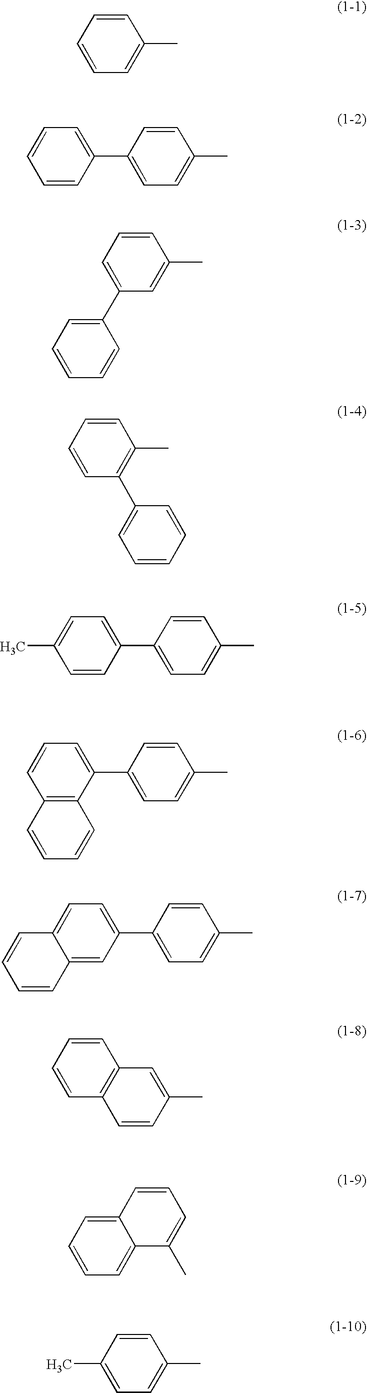

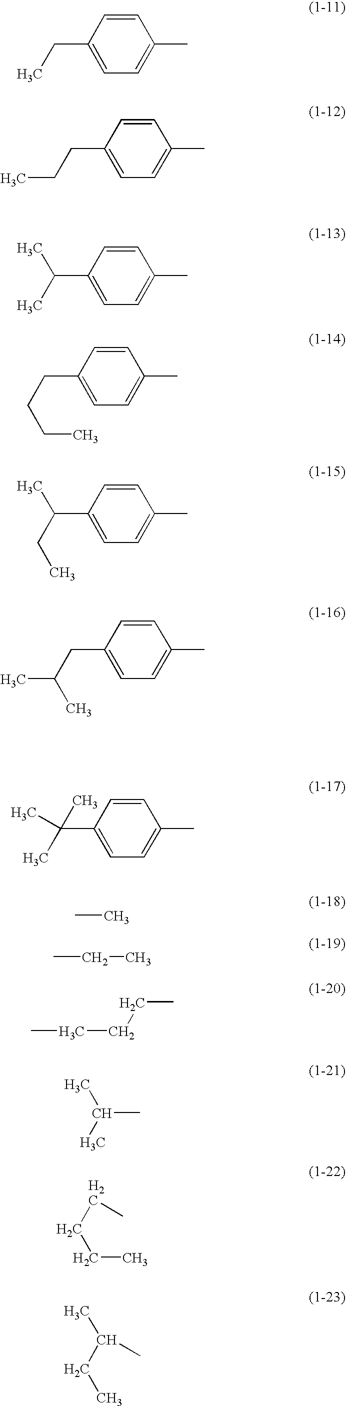

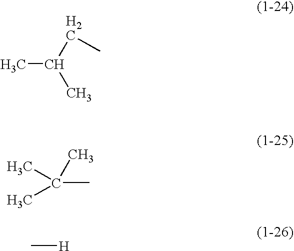

As specific structures of Ar¹ in General Formula (G1) and General Formula (G2), there are substituents represented by Structural Formulae (2-1) to (2-17).

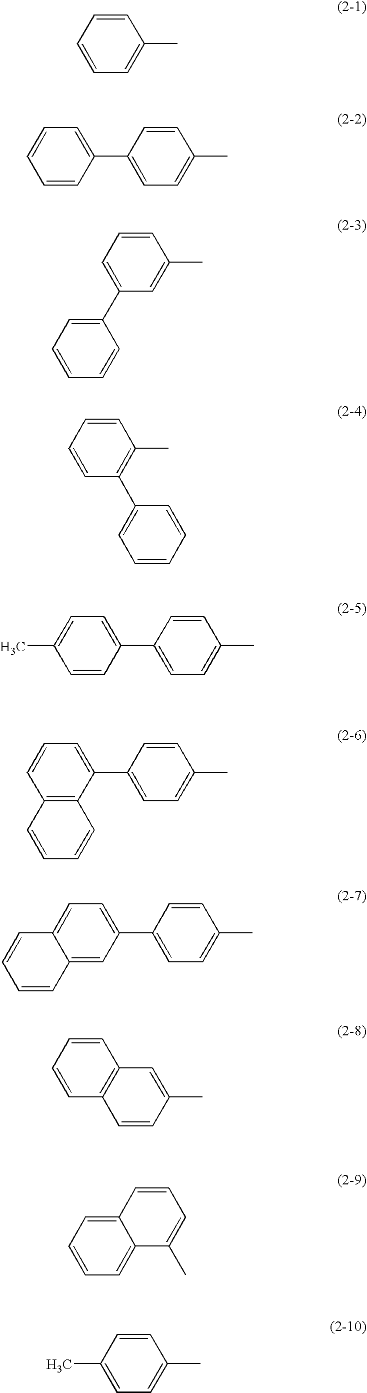

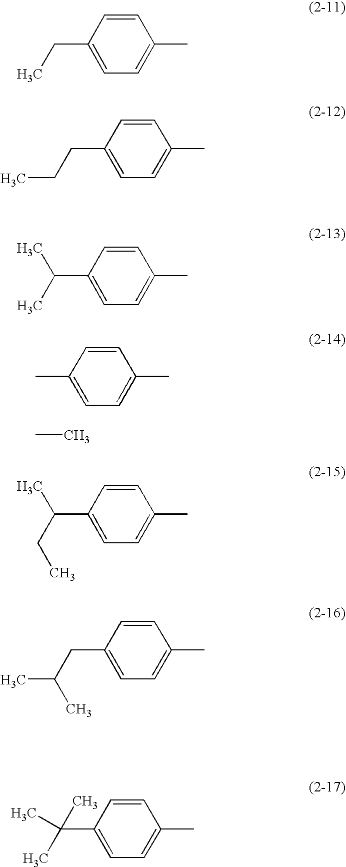

As specific structures of R¹¹ to R¹⁵ in General Formula (G2), there are substituents represented by Structural Formulae (3-1) to (3-15).

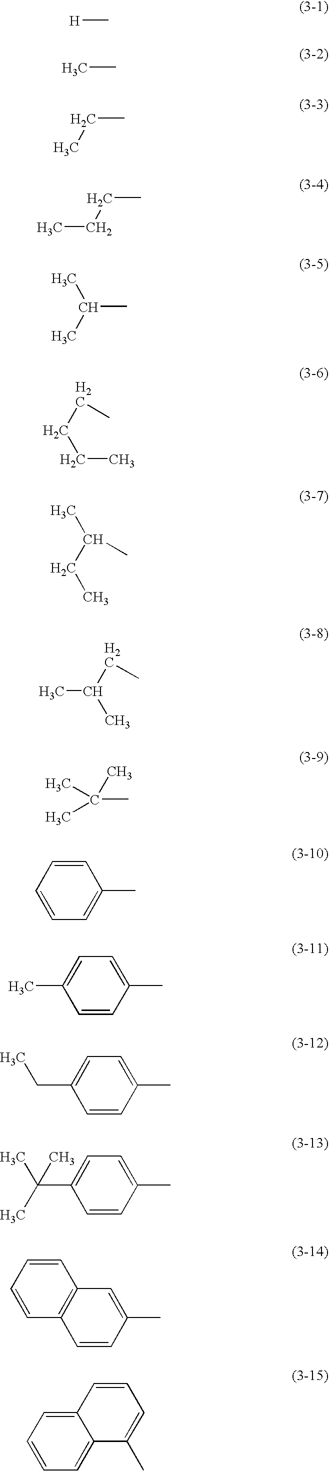

The oxadiazole derivatives represented by General Formula (G1) and General Formula (G2) are specifically exemplified by Structural Formulae (100) to (222). Note that the embodiments of the disclosed invention are not limited to these examples.

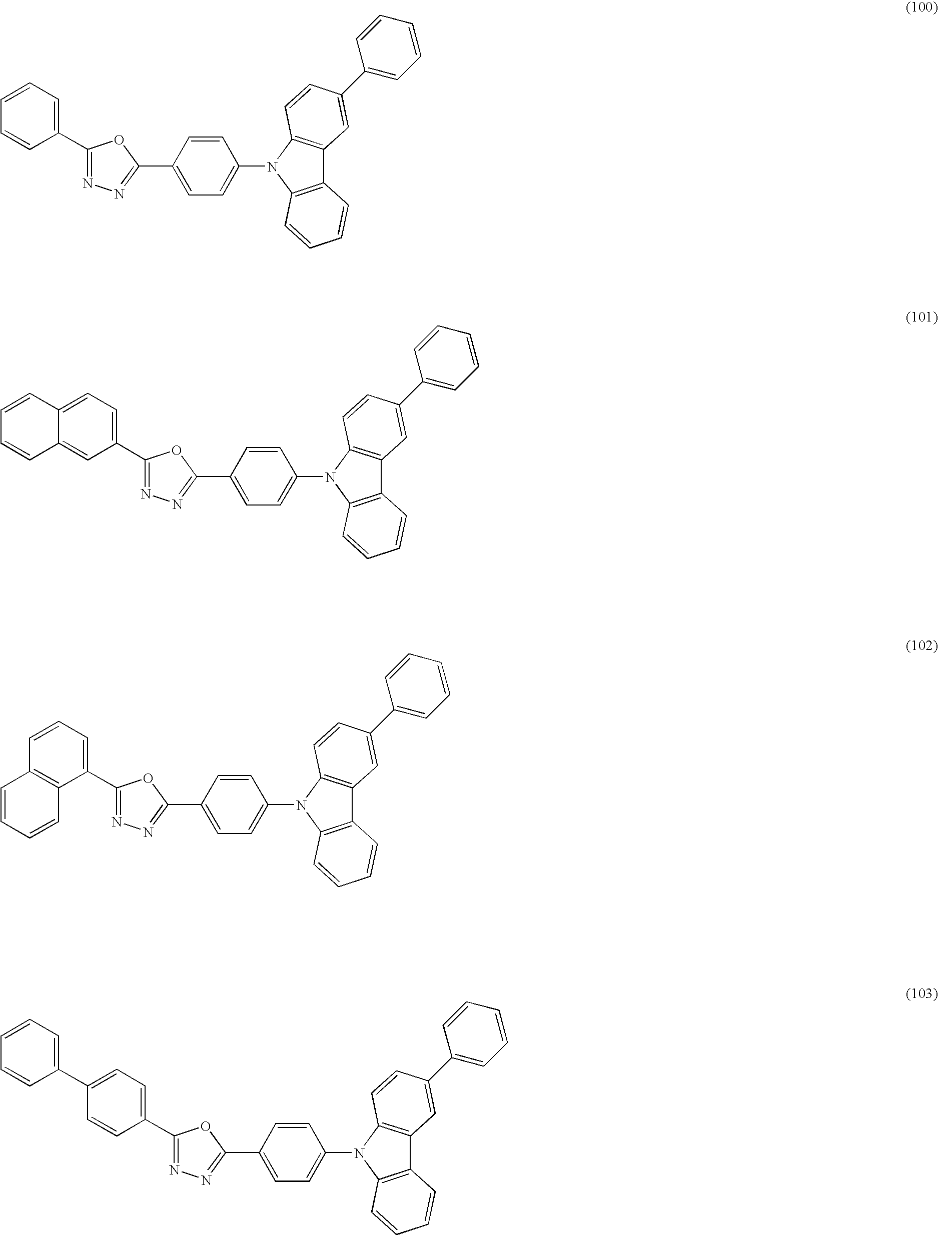

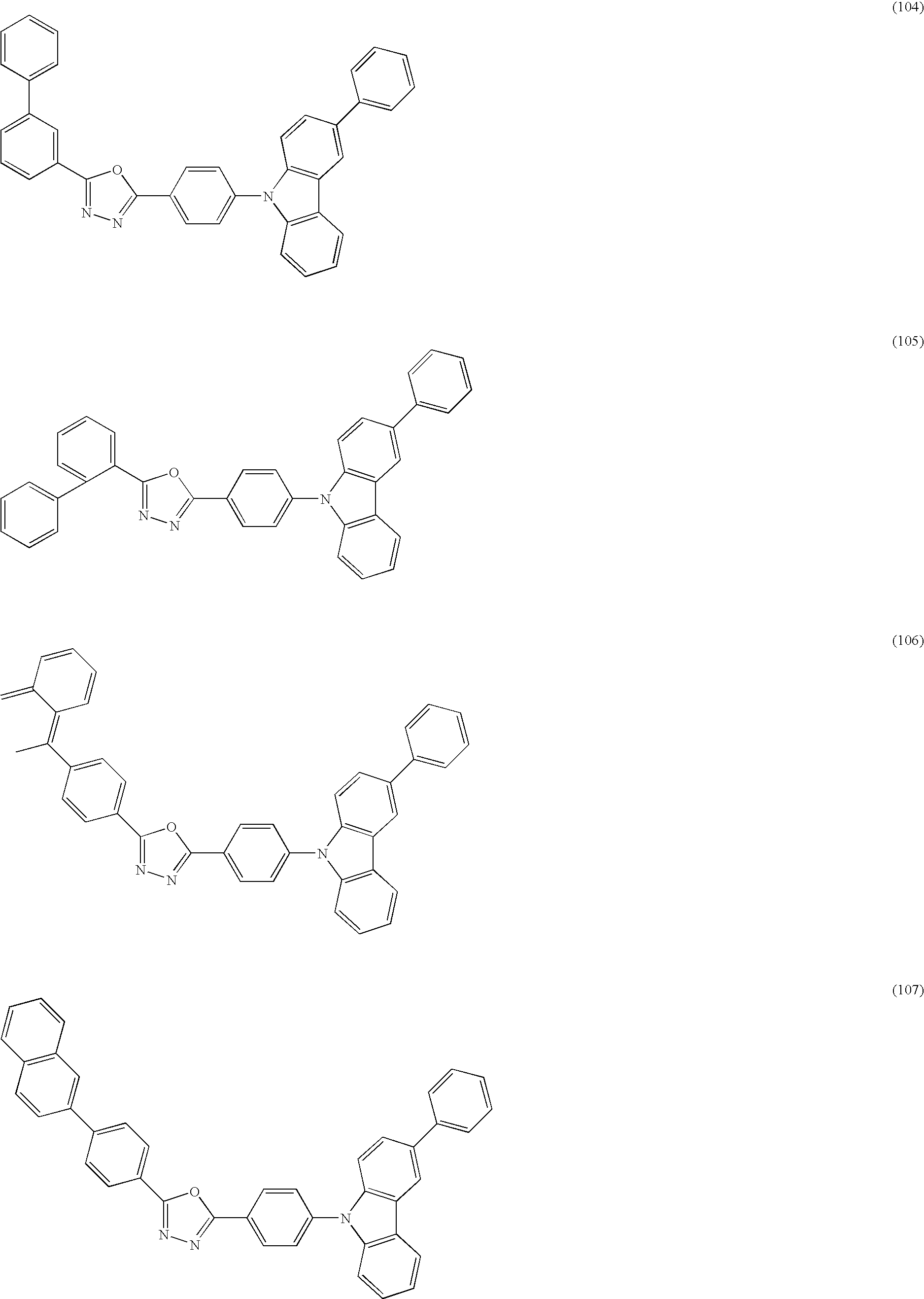

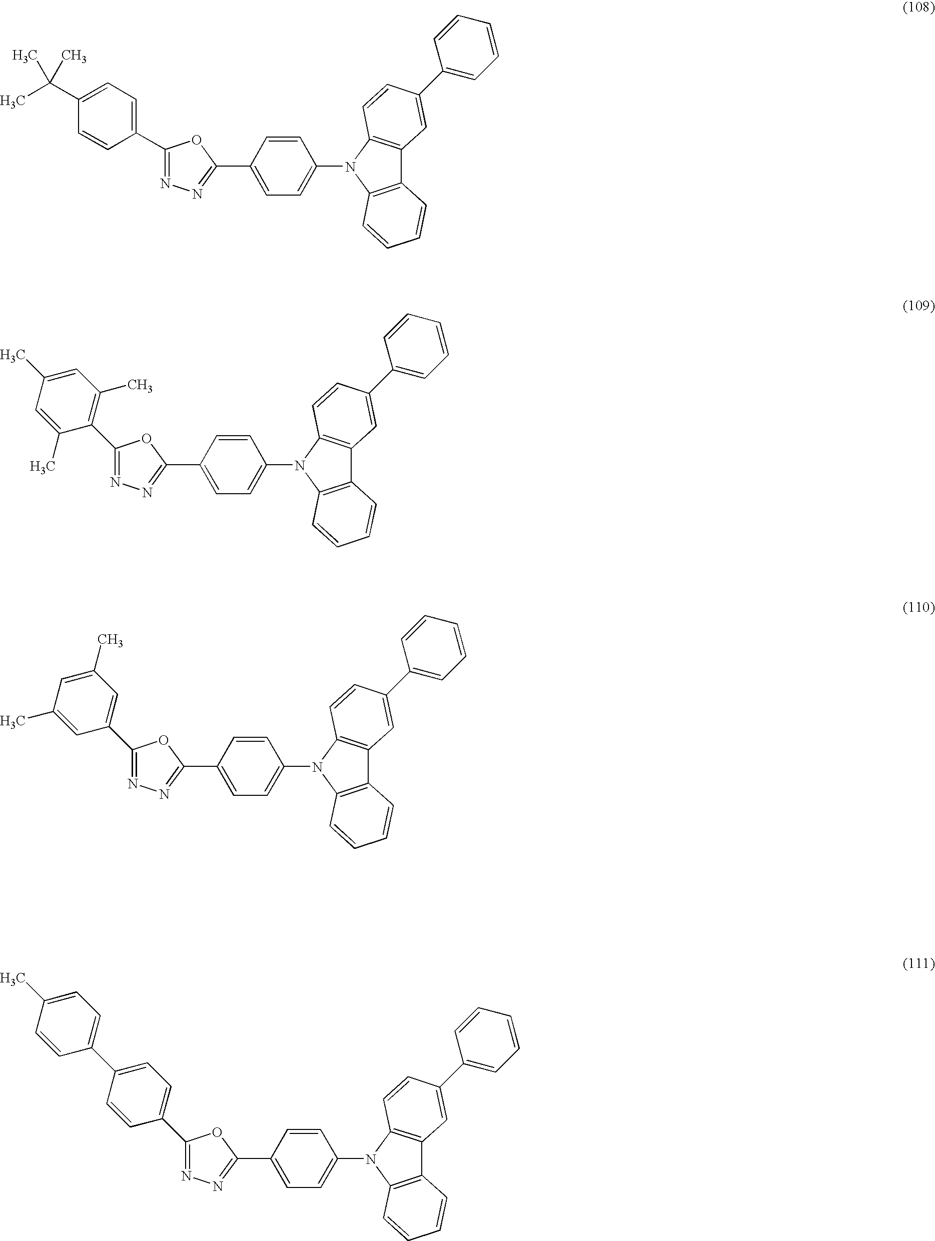

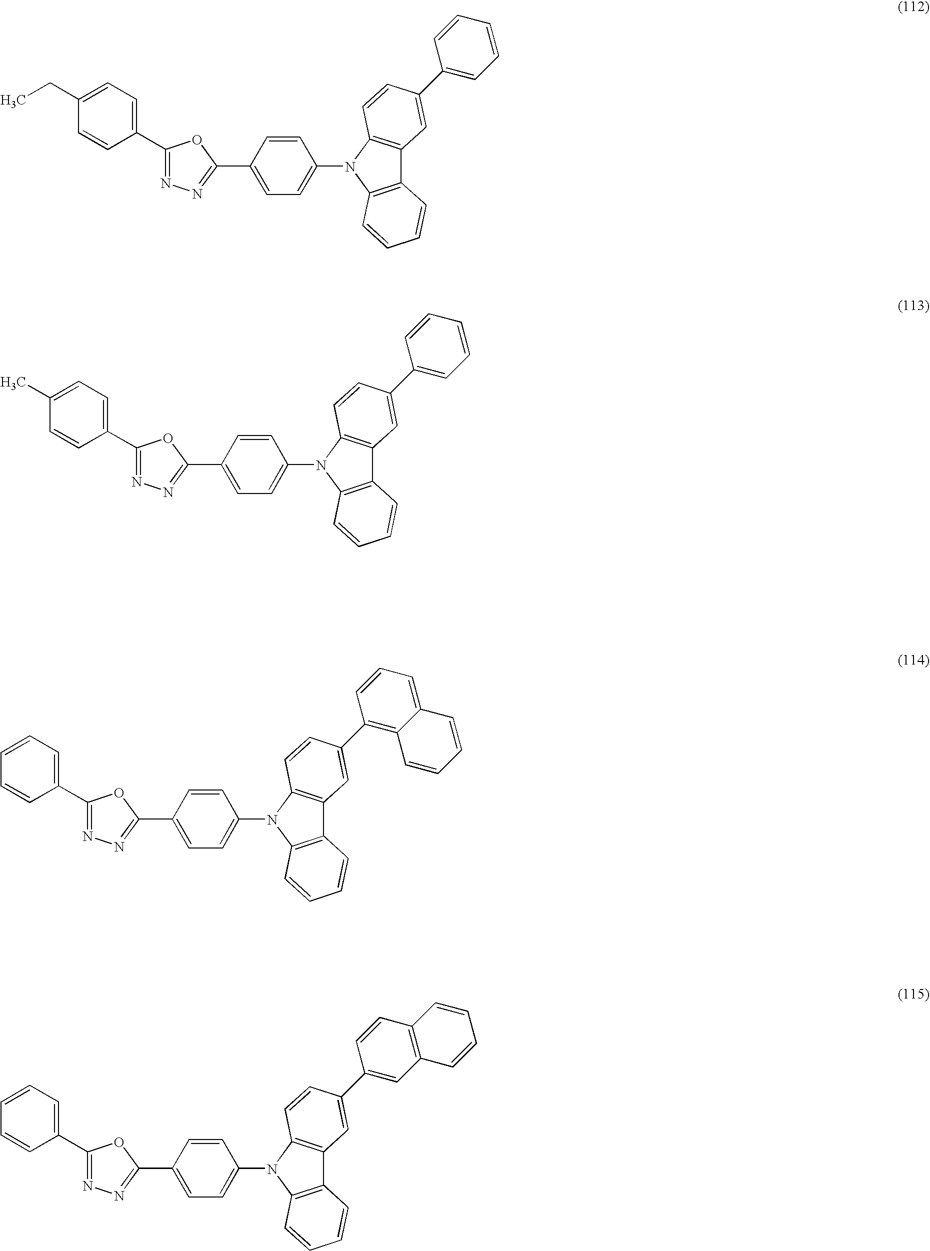

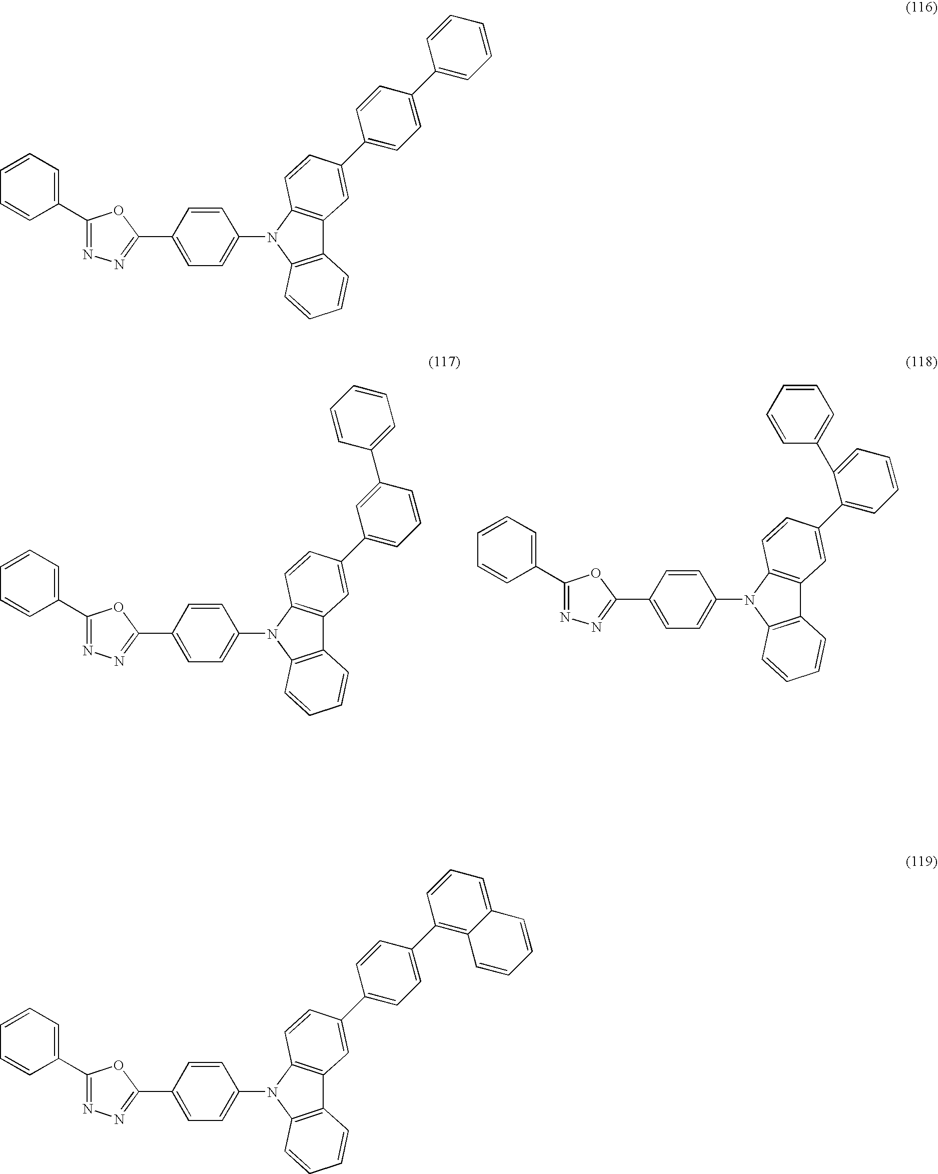

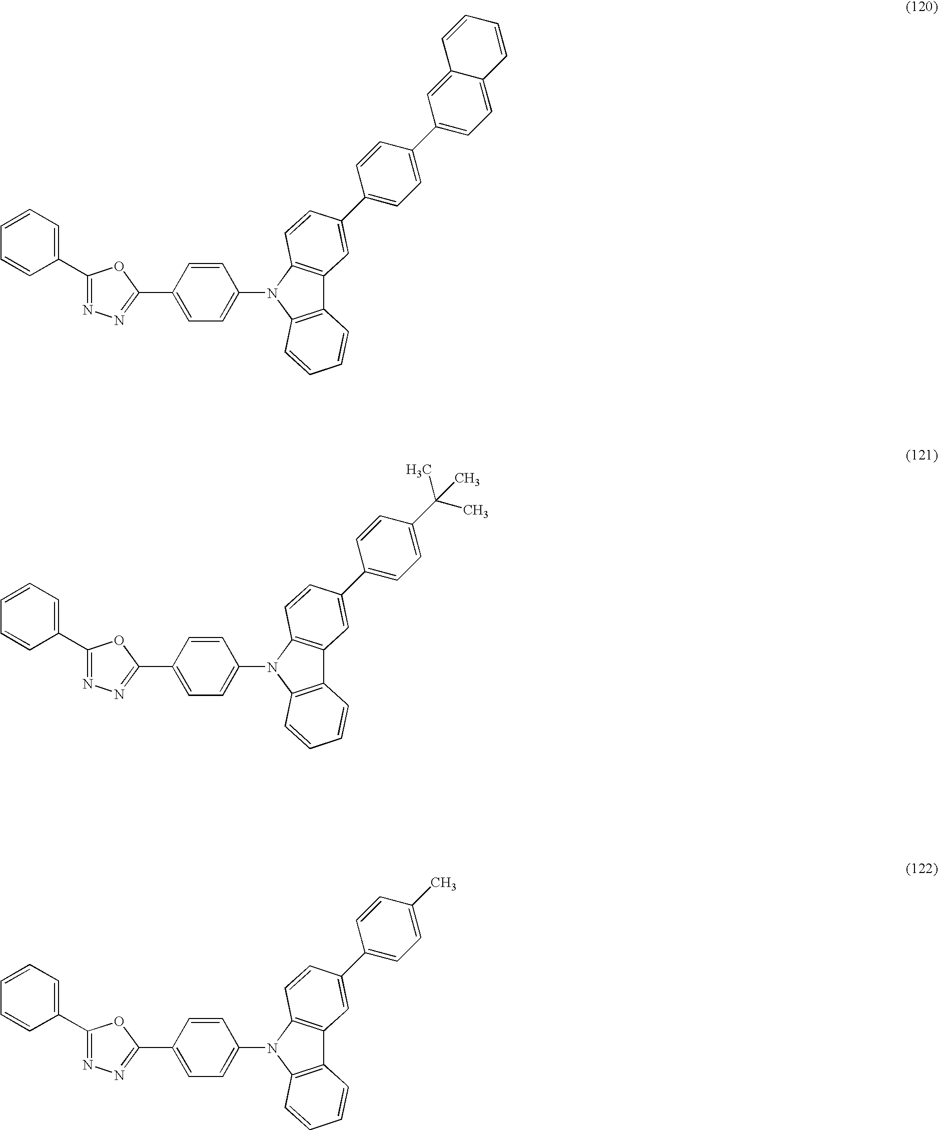

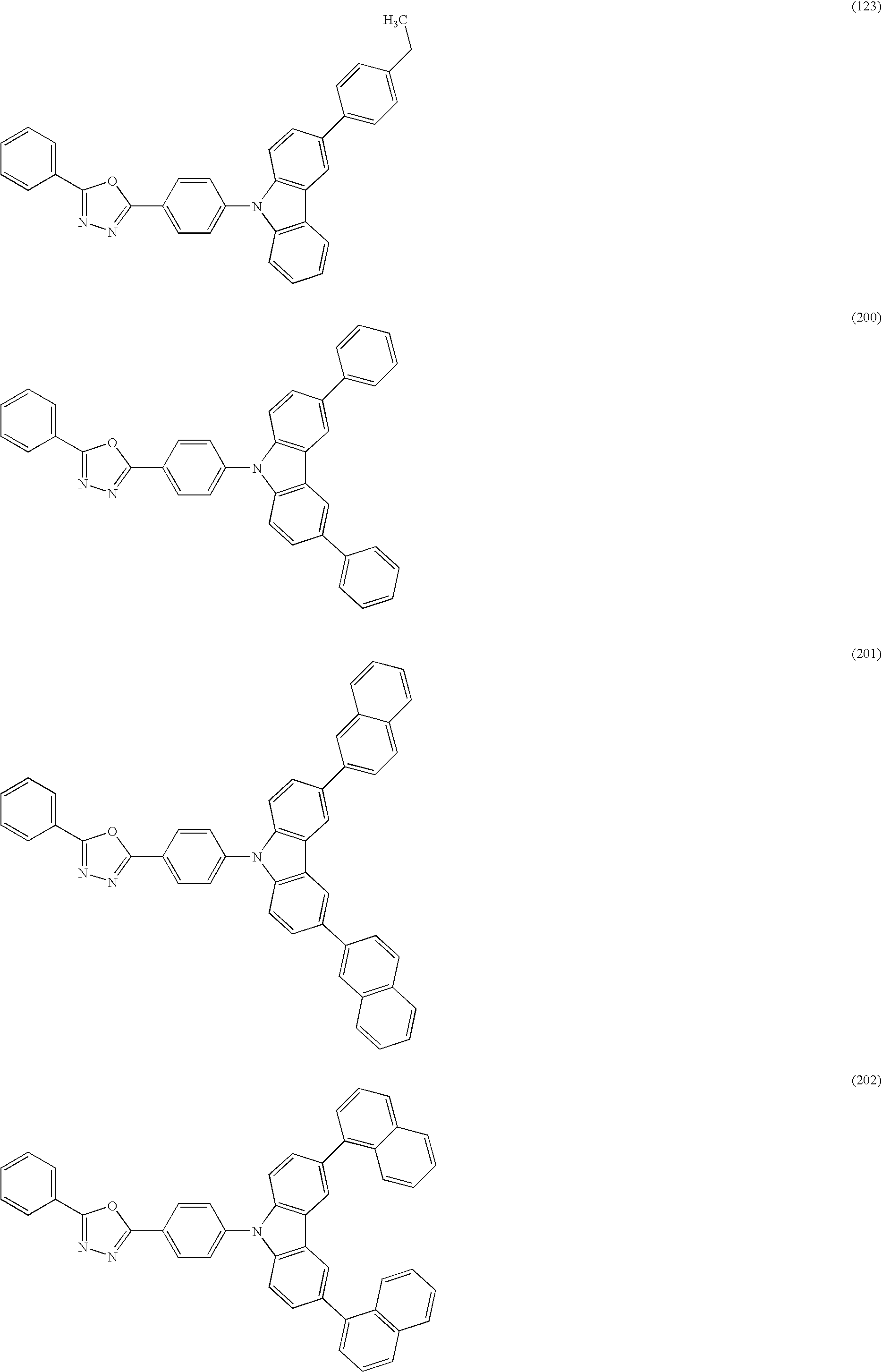

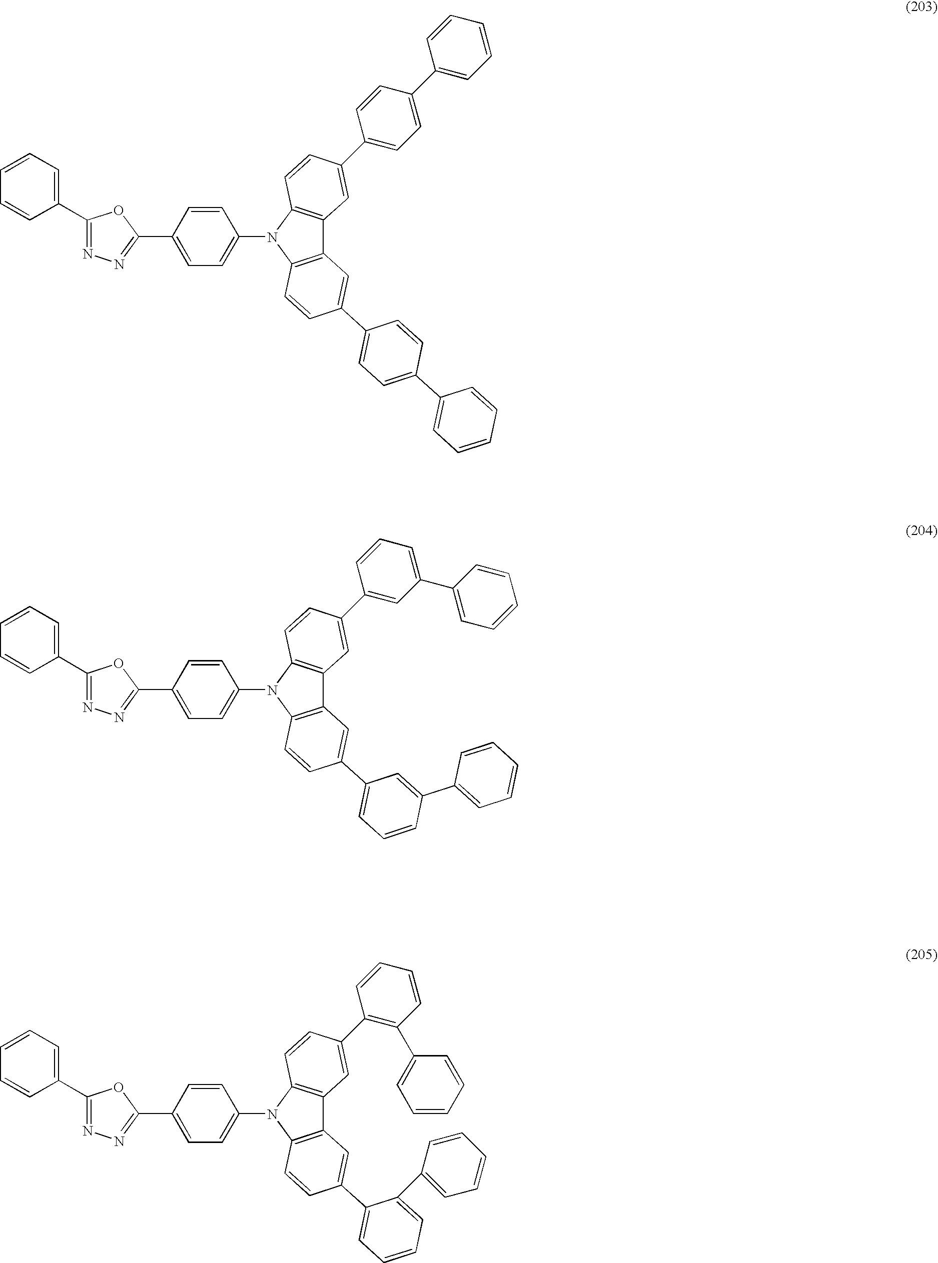

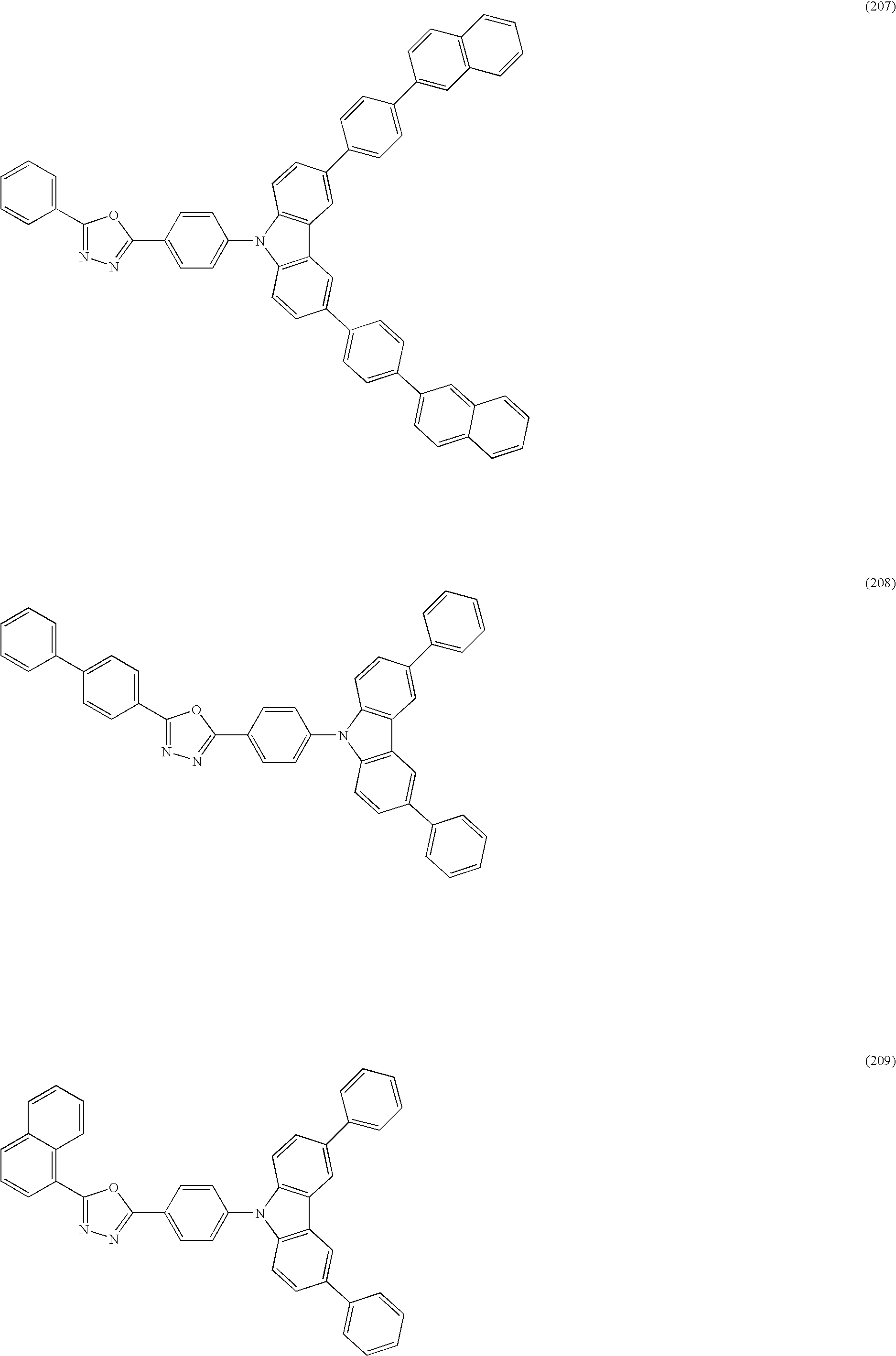

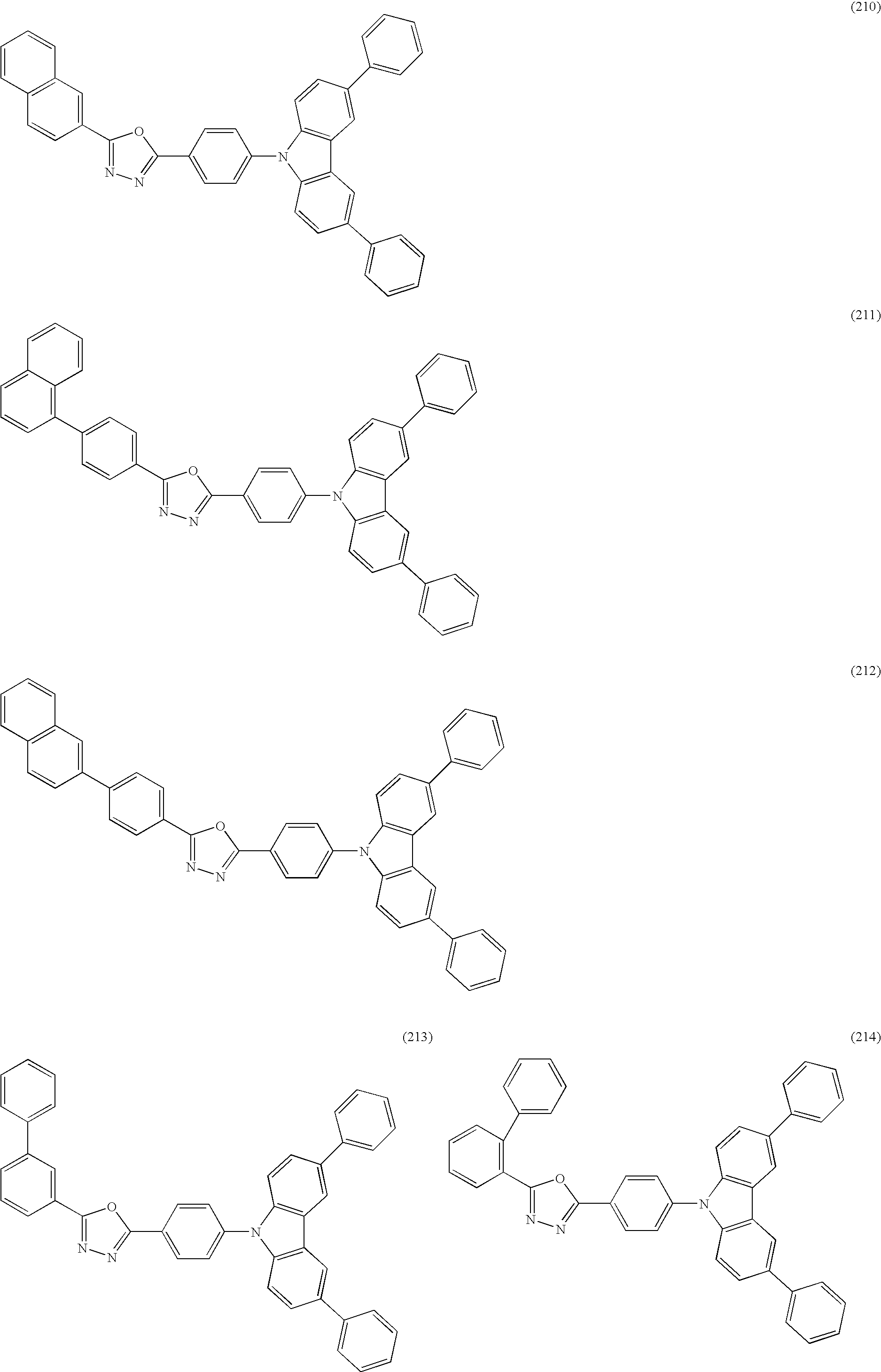

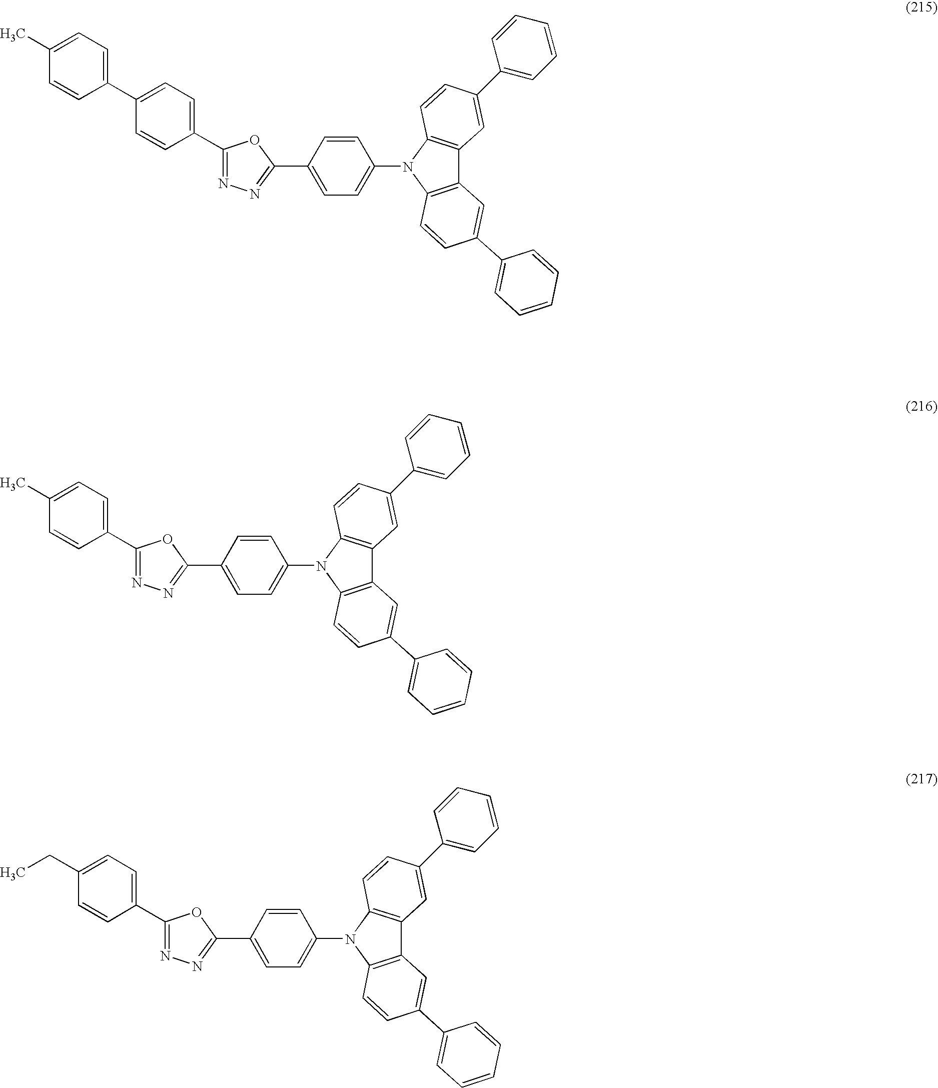

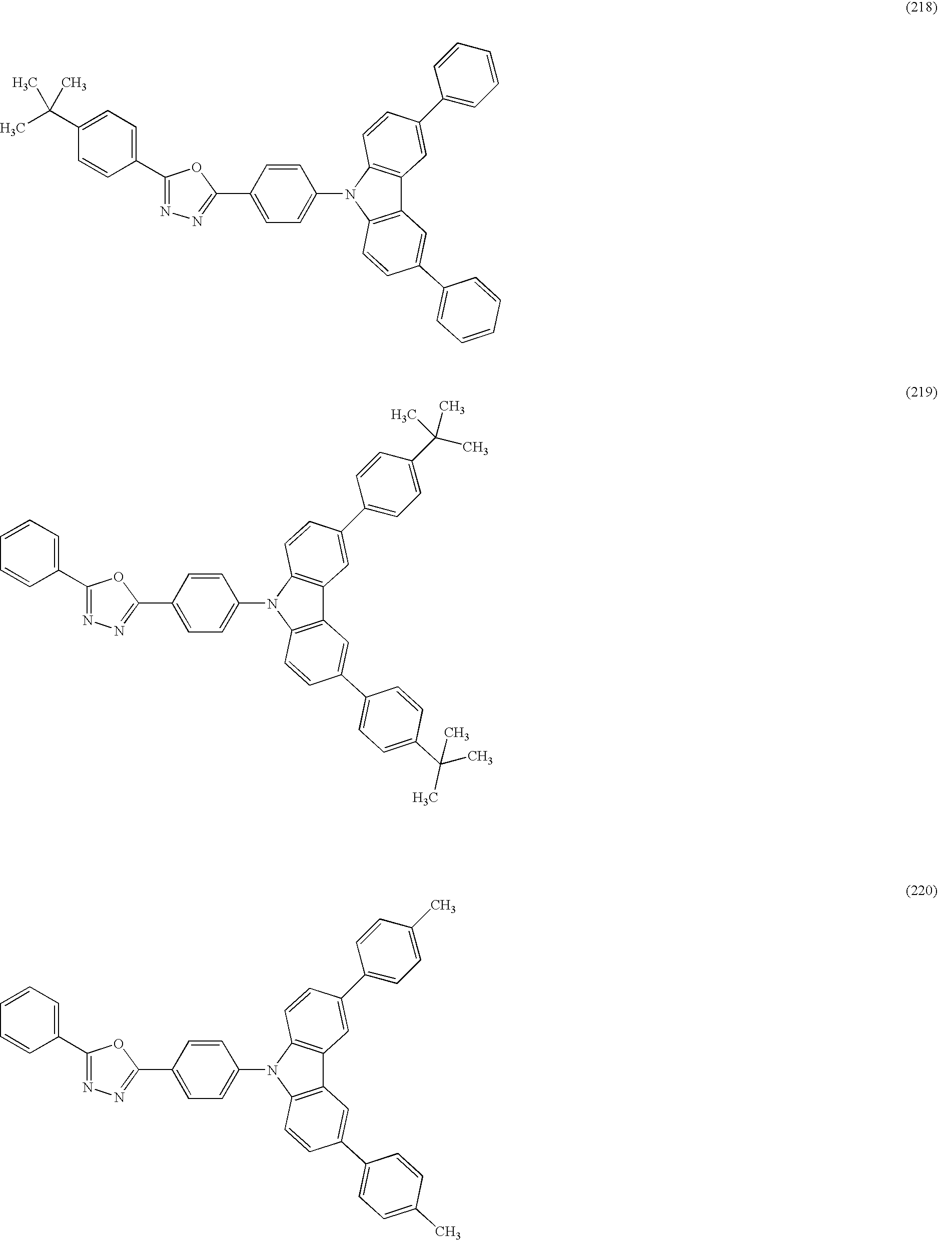

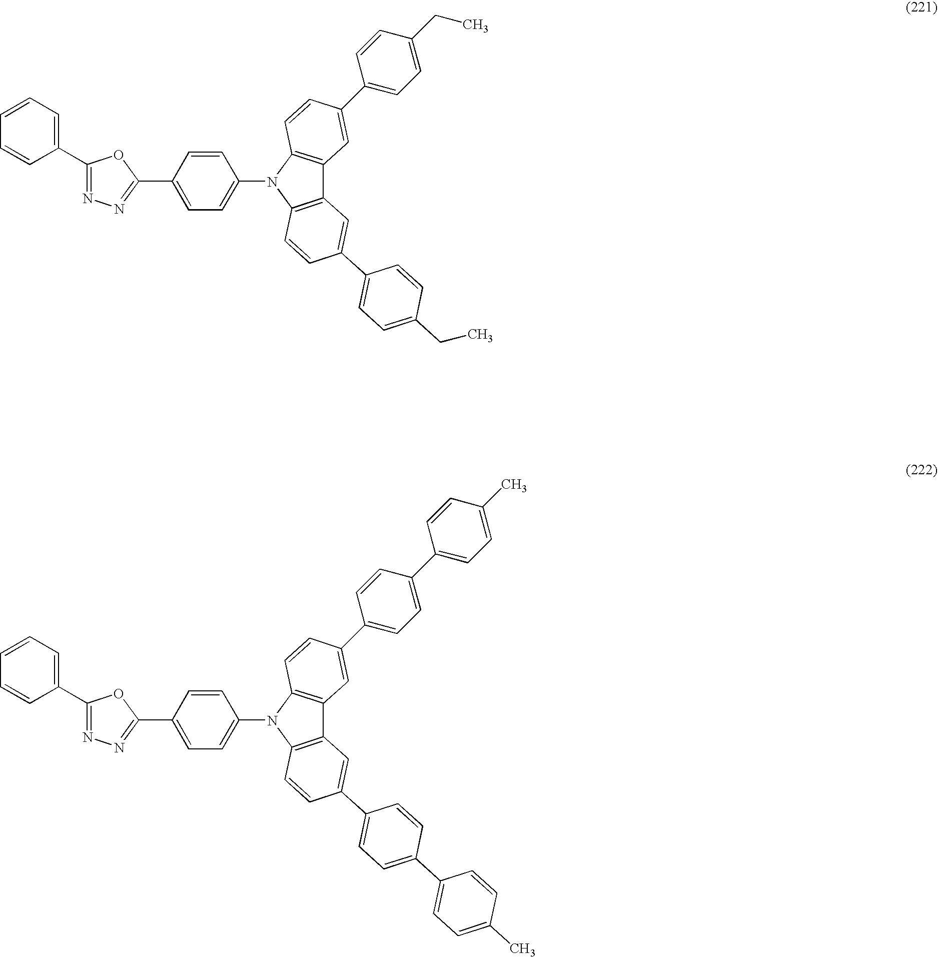

A variety of reactions can be applied to methods for synthesizing the oxadiazole derivatives which are one embodiment of the disclosed invention. For example, the oxadiazole derivative represented by General Formula (G1) can be synthesized by synthesis reactions described hereinbelow. Note that methods for synthesizing the oxadiazole derivatives are not limited to the following synthesis method.

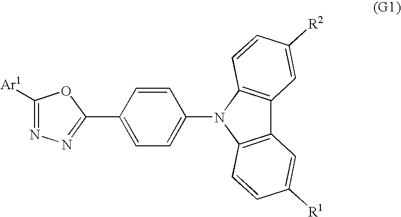

### <Method for Synthesizing Oxadiazole Derivative Represented by General Formula (G1)>

The oxadiazole derivative represented by General Formula (G1) can be synthesized according to Synthetic Scheme (A-1).

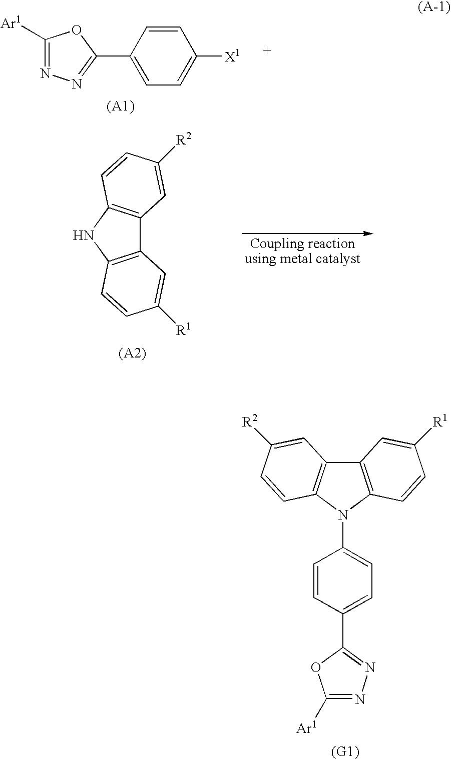

Specifically, the oxadiazole derivative (represented by General Formula (G1)) which are one embodiment of the disclosed invention can be obtained by coupling of a halide oxadiazole derivative (A1) with a 9H-carbazole derivative (A2) according to a Hartwig-Buchwald reaction using a palladium catalyst or according to an Ullmann reaction using copper or a copper compound. Note that the derivative represented by General Formula (G1) is referred to as an oxadiazole derivative in this specification and the like, but may be called a carbazole derivative.

In Synthetic Scheme (A-1), X¹ represents a halogen. The halogen is preferably iodine or bromine. In Synthetic Scheme (A-1), R¹ and R² independently represent a hydrogen atom, an alkyl group having 1 to 4 carbon atoms, or a substituted or unsubstituted aryl group having 6 to 10 carbon atoms. Note that at least one of R¹ and R² represents a substituted or unsubstituted aryl group having 6 to 10 carbon atoms in a ring. In addition, Ar¹ represents a substituted or unsubstituted aryl group having 6 to 10 carbon atoms in a ring. In R¹, R², and Ar¹, a substituent is an alkyl group having 1 to 4 carbon atoms or an aryl group having 6 to 10 carbon atoms in a ring.

In Synthetic Scheme (A-1), when a Hartwig-Buchwald reaction is carried out, examples of palladium catalysts that can be used include bis(dibenzylideneacetone)palladium(0), palladium(II) acetate, and the like. Examples of ligands of the palladium catalyst include tri(tert-butyl)phosphine, tri(n-hexyl)phosphine, tricyclohexylphosphine, and the like. Examples of bases usable here include an organic base such as sodium tert-butoxide, an inorganic base such as potassium carbonate, and the like. In addition, examples of solvents that can be used include toluene, xylene, benzene, tetrahydrofuran, and the like.

In Synthetic Scheme (A-1), when an Ullmann reaction is carried out, a copper compound such as copper(I) iodide or copper(II) acetate can be used. As an alternative to the copper compound, copper can be used. As bases usable here, an inorganic base such as potassium carbonate is given. Examples of solvents that can be used include 1,3-dimethyl-3,4,5,6-tetrahydro-2(1H)pyrimidinone (DMPU), toluene, xylene, benzene, and the like. In an Ullmann reaction, DMPU, xylene, or the like, which has a high boiling point, is preferably used because the desired substance can be obtained in a shorter time and a higher yield by setting the reaction temperature to 100° C. or more. In addition, setting the reaction temperature to 150° C. or more is further preferable, in which case DMPU or the like can be used.

The above description is one example of reaction schemes. The oxadiazole derivative (G1) which is one embodiment of the disclosed invention may be synthesized by any other synthesis method.

### Embodiment 2

In this embodiment, an example of a light-emitting element in which any of the oxadiazole derivatives described in the above embodiment is used for a light-emitting layer will be described with reference to the drawing.

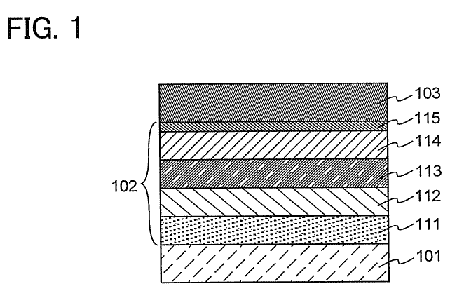

FIG. 1 illustrates an example of a light-emitting element in which an EL layer 102 including a light-emitting layer 113 is interposed between a first electrode 101 and a second electrode 103.

By applying a voltage to such a light-emitting element, holes injected from the first electrode 101 side and electrons injected from the second electrode 103 side recombine in the light-emitting layer 113, whereby a light-emitting organic compound is raised to an excited state. Then, the organic compound in the excited state emits light in transition to the ground state. Note that in the light-emitting element described in this embodiment, the first electrode 101 and the second electrode 103 function as an anode and a cathode, respectively. Further, in the structure illustrated in FIG. 1, the order of stacking layers may naturally be reversed.

For the first electrode 101 functioning as an anode is preferably formed using a metal, an alloy, an electrically conductive compound, a mixture thereof, or the like which has a high work function (specifically, a work function of 4.0 eV or more). Specific examples include indium oxide-tin oxide (ITO: indium tin oxide), indium oxide-tin oxide containing silicon or silicon oxide, indium oxide-zinc oxide (WO: indium zinc oxide), and indium oxide containing tungsten oxide and zinc oxide, and the like. Other than these, there are gold (Au), platinum (Pt), nickel (Ni), tungsten (W), chromium (Cr), molybdenum (Mo), iron (Fe), cobalt (Co), copper (Cu), palladium (Pd), titanium (Ti), and the like.

Note that, when a layer in contact with the first electrode 101 which is included in the EL layer 102 is formed using a composite material of an organic compound and an electron acceptor, a substance used for the first electrode 101 can be selected without being limited by the work function. For example, aluminum (Al), silver (Ag), an alloy including aluminum (e.g., AlSi), or the like can also be used.

Note that the first electrode 101 can be formed by, for example, a sputtering method, an evaporation method (including a vacuum evaporation method), or the like.

The EL layer 102 formed over the first electrode 101 has at least the light-emitting layer 113 and is formed to include any of the oxadiazole derivatives described in the above embodiment. The EL layer 102 can also include a known substance as a part, for which either a low molecular compound or a high molecular compound may be used. Note that the substances forming the EL layer 102 may include an inorganic compound as a part.

Further, as illustrated in FIG. 1, the EL layer 102 includes the light-emitting layer 113 and also the following layers stacked in appropriate combination: a hole-injection layer 111 including a substance having a high hole-injection property, a hole-transport layer 112 including a substance having a high hole-transport property, an electron-transport layer 114 including a substance having a high electron-transport property, an electron-injection layer 115 including a substance having a high electron-injection property, and the like.

The hole-injection layer 111 includes a substance having a high hole-injection property. As the substance having a high hole-injection property, a metal oxide such as molybdenum oxide, titanium oxide, vanadium oxide, rhenium oxide, ruthenium oxide, chromium oxide, zirconium oxide, hafnium oxide, tantalum oxide, silver oxide, tungsten oxide, or manganese oxide can be used. Alternatively, a phthalocyanine-based compound such as phthalocyanine (abbreviation: H₂Pc), copper(II) phthalocyanine (abbreviation: CuPc), or vanadyl phthalocyanine (abbreviation: VOPc) can be used.

Further, as examples of low molecular organic compounds, any of the following aromatic amine compounds can be used: 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[Ar-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1), and the like.

Further alternatively, any of high molecular compounds (e.g., oligomers, dendrimers, or polymers) can be used. For example, any of the following high molecular compounds can be used: poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), and poly[N,N′-bis(4-butylphenyl)-N,N′-bis(phenyl)benzidine] (abbreviation: Poly-TPD). Alternatively, a high molecular compound to which acid is added, such as poly(3,4-ethylenedioxythiophene)/poly(styrenesulfonic acid) (PEDOT/PSS), or polyaniline/poly(styrenesulfonic acid) (PAni/PSS), can be used.

Alternatively, for the hole-injection layer 111, a composite material formed by combining an organic compound and an electron acceptor may be used. Such a composite material has excellent hole-injection and -transport properties because the electron acceptor produces holes in the organic compound. In this case, as the organic compound, a material that can efficiently transport the produced holes (a substance having a high hole-transport property) is preferably used.

Note that an organic compound used for the above composite material preferably has a high hole-transport property. Specifically, a substance having a hole mobility of 10⁻⁶ cm²/Vs or more is preferably used. Further, this organic compound is not to be construed as being limited to such substances as long as it has a higher hole-transport property than an electron-transport property. Organic compounds that can be used for the composite material are specifically given below.

Examples of the organic compounds that can be used for the composite material include aromatic amine compounds such as TDATA, MTDATA, DPAB, DNTPD, DPA3B, PCzPCA1, PCzPCA2, PCzPCN1, 4,4′-bis[N-(1-naphthyl)-N-phenylamino]biphenyl (abbreviation: NPB), and N,N′-bis(3-methylphenyl)-N,N′-diphenyl-[1,1′-biphenyl]-4,4′-diamine (abbreviation: TPD) and carbazole derivatives such as 4,4′-di(N-carbazolyl)biphenyl (abbreviation: CBP), 1,3,5-tris[4-(N-carbazolyl)phenyl]benzene (abbreviation: TCPB), 9-[4-(N-carbazolyl)]phenyl-10-phenylanthracene (abbreviation: CzPA), and 1,4-bis[4-(N-carbazolyl)phenyl-2,3,5,6-tetraphenylbenzene.

Any of the following aromatic hydrocarbon compounds may be used: 2-tert-butyl-9,10-di(2-naphthyl)anthracene (abbreviation: t-BuDNA), 2-tert-butyl-9,10-di(1-naphthyl)anthracene, 9,10-bis(3,5-diphenylphenyl)anthracene (abbreviation: DPPA), 2-tert-butyl-9,10-bis(4-phenylphenyl)anthracene (abbreviation: t-BuDBA), 9,10-di(2-naphthyl)anthracene (abbreviation: DNA), 9,10-diphenylanthracene (abbreviation: DPAnth), 2-tert-butylanthracene (abbreviation: t-BuAnth), 9,10-bis(4-methyl-1-naphthyl)anthracene (abbreviation: DMNA), 9,10-bis[2-(1-naphthyl)phenyl)-2-tert-butyl-anthracene, 9,10-bis[2-(1-naphthyl)phenyl]anthracene, and 2,3,6,7-tetramethyl-9,10-di(1-naphthyl)anthracene.

Any of the following aromatic hydrocarbon compounds may also be used: 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, rubrene, perylene, 2,5,8,11-tetra(tert-butyl)perylene, pentacene, coronene, 4,4′-bis(2,2-diphenylvinyl)biphenyl (abbreviation: DPVBi), and 9,10-bis[4-(2,2-diphenylvinyl)phenyl]anthracene (abbreviation: DPVPA).

Further, as examples of electron acceptors that can be used for the composite material, there are organic compounds such as 7,7,8,8-tetracyano-2,3,5,6-tetrafluoroquinodimethane (abbreviation: F₄-TCNQ) and chloranil, transition metal oxides, and the like. Oxides of metals belonging to Group 4 to Group 8 of the periodic table may be used. For example, vanadium oxide, niobium oxide, tantalum oxide, chromium oxide, molybdenum oxide, tungsten oxide, manganese oxide, and rhenium oxide are suitable because of their high electron-accepting properties. Among these, molybdenum oxide is suitable because it is stable in air and its hygroscopic property is low so that it can be easily handled.

Note that a composite material formed using any of the above-mentioned high molecular compounds such as PVK, PVTPA, PTPDMA, and Poly-TPD and any of the above-mentioned electron acceptors may be used for the hole-injection layer 111.

The hole-transport layer 112 includes a substance having a high hole-transport property. As a substance having a high hole-transport property, there are aromatic amine compounds such as NPB, TPD, 4,4′-bis[N-(9,9-dimethylfluoren-2-yl)-N-phenylamino]biphenyl (abbreviation: DFLDPBi), and 4,4′-bis[N-(spiro-9,9′-bifluoren-2-yl)-N-phenylamino]biphenyl (abbreviation: BSPB). The substances mentioned here are mainly substances having a hole mobility of 10⁻⁶ cm²/Vs or more. However, any other substance may also be used as long as it has a higher hole-transport property than an electron-transport property. Note that the hole-transport layer 112 may have a single-layer structure or a stacked-layer structure.

Further alternatively, for the hole-transport layer 112, a high molecular compound such as PVK, PVTPA, PTPDMA, or Poly-TPD can be used.

The light-emitting layer 113 includes a substance having a high light-emitting property. Note that in this embodiment, description is given of an example in which any of the oxadiazole derivatives described in the above embodiment is used for the light-emitting layer. The above oxadiazole derivatives are suitably used as a host material in a light-emitting layer where a substance having a high light-emitting property (guest material) is dispersed in another substance (host material). However, embodiments of the disclosed invention are not to be construed as being limited to this structure. Any of the above oxadiazole derivatives may be used alone in the light-emitting layer.

In the case where any of the oxadiazole derivatives described in the above embodiment is used as a host material and a material that emits fluorescence is used as a guest material, it is preferable to use, as the guest material, a material whose lowest unoccupied molecular orbital (LUMO) level is lower and highest occupied molecular orbital (HOMO) level is higher than those of the oxadiazole derivative described in the above embodiment. Examples of materials for blue light emission include N,N-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), and the like. In addition, examples of materials for green light emission include N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), N-[9,10-bis(1,1′-biphenyl-2-yl)]-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), and the like. Further, examples of materials for yellow light emission include rubrene, 5,12-bis(1,1′-biphenyl-4-yl)-6,11-diphenyltetracene (abbreviation: BPT), and the like. Furthermore, examples of materials for red light emission include N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), and the like.

Alternatively, in the case where any of the oxadiazole derivatives described in the above embodiment is used as a host material and a material that emits phosphorescence is used as a guest material, a material having lower triplet excitation energy than the oxadiazole derivative described in the above embodiment is preferably used as the guest material. Examples of such materials include organometallic complexes such as bis[2-(3′,5′-bistrifluoromethylphenyl)pyridinato-N,C^{2′}]iridium(III)picolinate (abbreviation: Ir(CF₃ ppy)₂(pic)), bis[2-(4′,6′-difluorophenyl)pyridinato-N,C^{2′}]iridium(III) acetylacetonate (abbreviation: FIracac), tris(2-phenylpyridinato-N,C^{2′})iridium(III) (abbreviation: Ir(ppy)₃), bis(2-phenylpyridinato)iridium(III)acetylacetonato (abbreviation: Ir(ppy)₂(acac)), tris(acetylacetonato)(monophenanthroline)terbium(III) (abbreviation: Tb(acac)₃(Phen)), bis(benzo[h]quinolinato)iridium(III)acetylacetonate (abbreviation: Ir(bzq)₂(acac)), bis(2,4-diphenyl-1,3-oxazolato-N,C^{2′})iridium(III)acetylacetonate (abbreviation: Ir(dpo)₂(acac)), bis[2-(4′-perfluorophenylphenyl)pyridinato]iridium(III)acetylacetonate (abbreviation: Ir(p-PF-ph)₂(acac)), bis(2-phenylbenzothiazolato-N,C^{2′})iridium(III)acetylacetonate (abbreviation: Ir(bt)₂(acac)), bis[2-(2′-benzo[4,5-α]thienyl)pyridinato-N,C^{3′}]iridium(III) Ir(btp)₂(acac)), bis(1-phenylisoquinolinato-N,C^{2′})iridium(III) acetylacetonate (abbreviation: Ir(piq)₂(acac)), (acetylacetonato)bis[2,3-bis(4-fluorophenyl)quinoxalinato]iridium(III) (abbreviation: Ir(Fdpq)₂(acac)), (acetylacetonato)bis(2,3,5-triphenylpyrazinato)iridium(III) (abbreviation: Ir(tppr)₂(acac)), 2,3,7,8,12,13,17,18-octaethyl-21H,23H-porphyrin platinum(II) (abbreviation: PtOEP), tris(1,3-diphenyl-1,3-propanedionato)(monophenanthroline)europium(III) (abbreviation: Eu(DBM)₃(Phen)), and tris[1-(2-thenoyl)-3,3,3-trifluoroacetonato](monophenanthroline)europium(III) (abbreviation: Eu(TTA)₃(Phen)).

Since the oxadiazole derivatives described in the above embodiment have an electron-transport property, using any of them in a light-emitting layer achieves its high electron-transport property. In the light-emitting layer of such a structure, use of a guest material having a high electron-trapping property results in light emission with extremely high efficiency.

In addition, as a substance (host material) in which a light-emitting substance (guest material) is dispersed, plural kinds of substances can be used. Therefore, the light-emitting layer may include a second host material in addition to the oxadiazole derivatives described in the above embodiment.

Further, as a light-emitting substance, any of the above oxadiazole derivatives can be used alone or as a guest material.

The electron-transport layer 114 includes a substance having a high electron-transport property. For the electron-transport layer 114, it is possible to use a metal complex such as Alq₃, tris(4-methyl-8-quinolinolato)aluminum (abbreviation: Almq₃), bis(10-hydroxybenzo[h]quinolinato)beryllium (abbreviation: BeBq₂), BAlq, Zn(BOX)₂, or bis[2-(2′-hydroxyphenyl)benzothiazolato]zinc(II) (abbreviation: Zn(BTZ)₂). Alternatively, it is possible to use a heteroaromatic compound such as 2-(4-biphenyl)-1)-5-(4-tert-butylphenyl)-1,3,4-oxadiazole (abbreviation: PBD), 1,3-bis[5-(p-tert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), or 4,4′-bis(5-methylbenzoxazole-2-yl)stilbene (abbreviation: BzOs). Further alternatively, a high molecular compound such as poly(2,5-pyridinediyl) (abbreviation: PPy), poly[(9,9-dihexylfluorene-2,7-diyl)-co-(pyridine-3,5-diyl)] (abbreviation: PF-Py), or poly[(9,9-dioctylfluorene-2,7-diyl)-co-(2,2′-bipyridine-6,6′-diyl)] (abbreviation: PF-BPy) may be used. The substances described here are mainly substances having electron mobility of 10⁻⁶ cm²/Vs or more. Note that a substance other than the above substances may be used as long as it has a higher electron-transport property than a hole-transport property.

In addition, the electron-transport layer 114 may have a single-layer structure or a stacked-layer structure.

The electron-injection layer 115 includes a substance having a high electron-injection property. For the electron-injection layer 115, an alkali metal, an alkaline earth metal, or a compound thereof, such as lithium fluoride (LW), cesium fluoride (CsF), calcium fluoride (CaF₂), or lithium oxide (LiOₓ) can be used. Alternatively, a rare earth metal compound such as erbium fluoride (ErF₃) can also be used. Further alternatively, any of the above-described substances that are used to form the electron-transport layer 114 may be used.

For the electron-injection layer 115, a composite material formed by combining an organic compound and an electron donor may be used. Such a composite material has excellent electron-injection and -transport properties because the electron donor produces electrons in the organic compound. In this case, as the organic compound, a material that can efficiently transport the produced electrons is preferably used: for example, any of the above-described substances that are used to form the electron-transport layer 114 can be used. As the electron donor, a substance exhibiting an electron-donating property to the organic compound is used. Specifically, it is preferable to use any of alkali metals, alkali earth metals, or rare earth metals, such as lithium, cesium, magnesium, calcium, erbium, ytterbium, or the like. Alternatively, it is preferable to use any of alkali metal oxides or alkaline earth metal oxides: lithium oxide, calcium oxide, barium oxide, or the like. A Lewis base such as magnesium oxide can also be used. Alternatively, an organic compound such as tetrathiafulvalene (abbreviation: TTF) can be used.

Note that the hole-injection layer 111, hole-transport layer 112, light-emitting layer 113, electron-transport layer 114, and electron-injection layer 115 which are described above can each be formed by an evaporation method (including a vacuum evaporation method), and an inkjet method, a coating method, or the like.

The second electrode 103 functioning as a cathode is preferably formed using a metal, an alloy, an electrically conductive compound, a mixture thereof; or the like which has a low work function (preferably, 3.8 eV or lower), or the like. Specifically, any of the following materials can be used: Al, silver, and the like, as well as elements that belong to Group 1 or Group 2 of the periodic table, that is, alkali metals such as lithium (Li) and cesium (Cs) or alkaline earth metals such as magnesium (Mg), calcium (Ca), and strontium (Sr), or alloys thereof (e.g., MgAg and AlLi); rare earth metals such as europium (Eu) and ytterbium (Yb), or alloys thereof.

Note that, when a layer in contact with the second electrode 103 which is included in the EL layer 102 is formed using the above-described composite material of the organic compound and the electron donor, a material used for the second electrode 103 can be selected without being limited by the work function. For example, any of a variety of conductive materials such as Al, Ag, ITO, and indium oxide-tin oxide containing silicon or silicon oxide can be used.

In the formation of the second electrode 103, a vacuum evaporation method or a sputtering method can be used. Alternatively, when a silver paste or the like is used, a coating method, an inkjet method, or the like may be used.

In the above-described light-emitting element of the present invention, holes and electrons generated by a potential difference between the first electrode 101 and the second electrode 103 recombine in the EL layer 102, thereby emitting light. Then, this emitted light is extracted out through either the first electrode 101 or the second electrode 103, or both. Accordingly, either the first electrode 101 or the second electrode 103 or both have a light-transmitting property.

Note that with the use of the light-emitting element described in this embodiment, a passive matrix light-emitting device or an active matrix light-emitting device in which drive of the light-emitting element is controlled by a thin film transistor (TFT) can be fabricated.

Note that there is no particular limitation on the structure of the TFT in the case of fabricating an active matrix light-emitting device. Further, either an n-type TFT or a p-type TFT may be used. Furthermore, there is no particular limitation on a semiconductor material used for the TFT. For example, any of the following materials can be used: silicon-based semiconductor materials (which may be amorphous, crystalline, or single crystal), germanium-based semiconductor materials, chalcogenide-based semiconductor materials, or other variety of semiconductor materials. Obviously, an oxide semiconductor material may be used.

In this embodiment, any of the above-described oxadiazole derivatives is used to form the light-emitting layer 113. Accordingly, a light-emitting element with high power efficiency and long lifetime can be provided.

Note that this embodiment can be used in appropriate combination with any structure described in the above embodiment.

### Embodiment 3

The light-emitting element which is one embodiment of the disclosed invention may have a plurality of light-emitting layers. By producing light emission from each light-emitting layer, light which is a combination thereof can be obtained. White light emission can thus be obtained, for example. In this embodiment, one embodiment of a light-emitting element having a plurality of light-emitting layers is described with reference to the drawing.

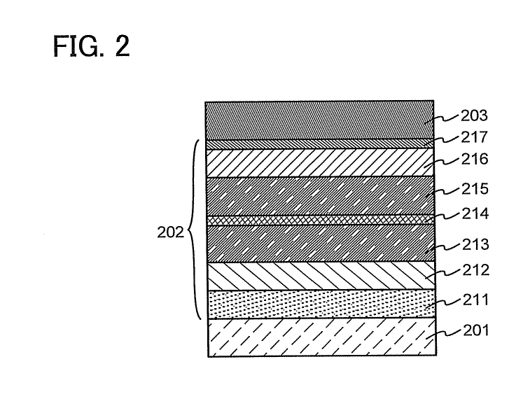

In FIG. 2, an EL layer 202 including a first light-emitting layer 213 and a second light-emitting layer 215 is provided between a first electrode 201 and a second electrode 203 to enable emission of light that is a combination of light emitted from the first light-emitting layer 213 and light emitted from the second light-emitting layer 215. A separation layer 214 is preferably formed between the first light-emitting layer 213 and the second light-emitting layer 215.

By application of a voltage such that the potential of the first electrode 201 is higher than that of the second electrode 203, a current flows between the first electrode 201 and the second electrode 203, and holes and/or electrons move to the first light-emitting layer 213, the second light-emitting layer 215, and the separation layer 214. Accordingly, a first light-emitting substance included in the first light-emitting layer 213 and a second light-emitting substance included in the second light-emitting layer 215 are raised to an excited state. Then, the light-emitting substances in the excited state emit light in transition to the ground state.

The first light-emitting layer 213 includes the first light-emitting substance typified by a fluorescent compound such as perylene, 2,5,8,11-tetra(tert-butyl)perylene (abbreviation: TBP), DPVBi, 4,4′-bis[2-(N-ethylcarbazol-3-yl)vinyl]biphenyl (abbreviation: BCzVBi), BAlq, or bis(2-methyl-8-quinolinolato)galliumchloride (abbreviation: Gamq₂Cl) or a phosphorescent compound such as bis{2-[3,5-bis(trifluoromethyl)phenyl]pyridinato-N,C²}iridium(III) picolinate (abbreviation: Ir(CF₃ ppy)₂(pic)), bis[2-(4′,6′-difluorophenyl)pyridinato-N,C^{2′}]iridium(III) acetylacetonate (abbreviation: FIr(acac)), bis[2-(4′,6′-difluorophenyl)pyridinato-N,C^{2′}]iridium(M) picolinate (abbreviation: FIrpic), or bis[2-(4′,6′-difluorophenyl)pyridinato-N,C^{2′}]iridium(III) tetra(1-pyrazolyl)borate (abbreviation: FIr6), from which light emission with a peak at 450 to 510 nm in an emission spectrum (i.e., blue light to blue green light) can be obtained.

When a fluorescent compound is used as the first light-emitting substance, the first light-emitting layer 213 preferably has a structure in which a substance having larger singlet excited energy than that of the first light-emitting substance is used as a first host and the first light-emitting substance is dispersed as a guest. Alternatively, when a phosphorescent compound is used as the first light-emitting substance, the first light-emitting layer 213 preferably has a structure in which a substance having larger triplet excited energy than that of the first light-emitting substance is used as a first host and the first light-emitting substance is dispersed as a guest. As the first host, NPB, CBP, TCTA, or the like, which is described above, or DNA, t-BuDNA, or the like can be used. Note that the singlet excitation energy is referred to as an energy difference between a ground state and a singlet excited state. In addition, the triplet excitation energy is referred to as an energy difference between a ground state and a triplet excited state.

Further, the second light-emitting layer 215 includes any of the oxadiazole derivatives described in Embodiment 1. The structure of the second light-emitting layer 215 is similar to that of the light-emitting layer 113 which is described in Embodiment 2.

In addition, the separation layer 214 can be formed using TPAQn, NPB, CBP, TCTA, Znpp₂, ZnBOX or the like described above, specifically. Provision of such a separation layer 214 can prevent an undesirable increase in the emission intensity of only either the first light-emitting layer 213 or the second light-emitting layer 215. Note that the separation layer 214 is not a necessary component. For example, the separation layer 214 may be provided in the case where the ratio of the emission intensity of the first light-emitting layer 213 to that of the second light-emitting layer 215 needs to be adjusted. Further, any oxadiazole derivative which is one embodiment of the disclosed invention may be used for the separation layer 214.

Note that in this embodiment, any of the oxadiazole derivatives described in the above embodiment is used for the second light-emitting layer 215, while another light-emitting substance is used for the first light-emitting layer 213. However, any of oxadiazole derivatives described in the above embodiment may be used for the first light-emitting layer 213, while another light-emitting substance may be used for the second light-emitting layer 215.

Further, although a light-emitting element including two light-emitting layers is described in this embodiment, the number of the light-emitting layers is not limited to two and may be three or more.

Note that the first electrode 201 has a structure similar to that of the first electrode 101 which is described in the above embodiment. Also, the second electrode 203 has a structure similar to that of the second electrode 103 which is described in the above embodiment.

Further, in this embodiment, description is given of an example in which a hole-injection layer 211, a hole-transport layer 212, an electron-transport layer 216, and an electron-injection layer 217 are provided. These layers can have a structure similar to that described in the above embodiment. However, they are not necessary components. These layers are provided according to element characteristics.

Note that this embodiment can be used in appropriate combination with any structure described in the above embodiments.

### Embodiment 4

In this embodiment, a light-emitting element having a plurality of EL layers (hereinafter, referred to as a stacked-type element) is described with reference to the drawing.

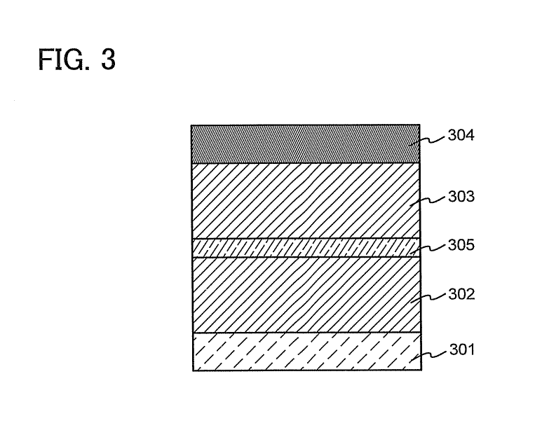

FIG. 3 illustrates a stacked-type light-emitting element that has a plurality of EL layers (a first EL layer 302 and a second EL layer 303) between a first electrode 301 and a second electrode 304. Note that although a structure in which two EL layers are formed is described in this embodiment, three or more EL layers may be formed.

In this embodiment, the first electrode 301 functions as an anode, and the second electrode 304 functions as a cathode. Note that the first electrode 301 and the second electrode 304 can have structures similar to those described in the above embodiments. Further, although the plurality of EL layers (the first EL layer 302 and the second EL layer 303) may be formed as described in the above embodiments, either layer may have a structure different from that described in the above embodiments. That is, the structures of the first EL layer 302 and the second EL layer 303 may be the same or different from each other.

Further, a charge generation layer 305 is provided between the plurality of EL layers (the first EL layer 302 and the second EL layer 303). The charge generation layer 305 has a function of injecting electrons into one of the EL layers and injecting holes into the other of the EL layers when a voltage is applied to the first electrode 301 and the second electrode 304. In this embodiment, when a voltage is applied so that the potential of the first electrode 301 is higher than that of the second electrode 304, the charge generation layer 305 injects electrons into the first EL layer 302 and injects holes into the second EL layer 303.

Note that the charge generation layer 305 preferably has a light-transmitting property in terms of light extraction efficiency. Further, the electric conductivity of the charge generation layer 305 may be lower than that of the first electrode 301 or the second electrode 304.

The charge generation layer 305 may have either a structure in which an electron acceptor is added to a substance having a high hole-transport property or a structure in which a substance having an electron donor is added to a substance having a high electron-transport property. Alternatively, both of these structures may be stacked.

The description in the above embodiment can be referred to for details of the organic compound having a high hole-transport property and the electron acceptor. Also, the description in the above embodiment can be referred to for details of the organic compound having a high electron-transport property and the electron donor.

Forming the charge generation layer 305 by using the above materials can suppress an increase in drive voltage which is caused by the stack of the EL layers.

By an arrangement in which the charge generation layer partitions the plurality of EL layers, as in the light-emitting element according to this embodiment, luminance can be improved while current density is kept low. Thus, a light-emitting element that can emit light with high luminance and has long lifetime can be achieved.

Further, by forming the EL layers to emit light of different colors from each other, an emission color that is provided by the light-emitting element as a whole can be controlled. For example, by forming a light-emitting element having two EL layers such that the emission color of the first EL layer and the emission color of the second EL layer are complementary colors, the light-emitting element can provide white light emission as a whole. Note that “complementary colors” refer to colors that can produce an achromatic color when mixed. In other words, when light of complementary colors is mixed, white light emission can be obtained. This can be applied to a light-emitting element having three or more EL layers.

Note that this embodiment can be used in appropriate combination with any structure described in the above embodiment.

### Embodiment 5

In this embodiment, description is made of a passive-matrix light-emitting device and an active light-emitting device each of which uses a light-emitting element, as one embodiment of the disclosed invention.

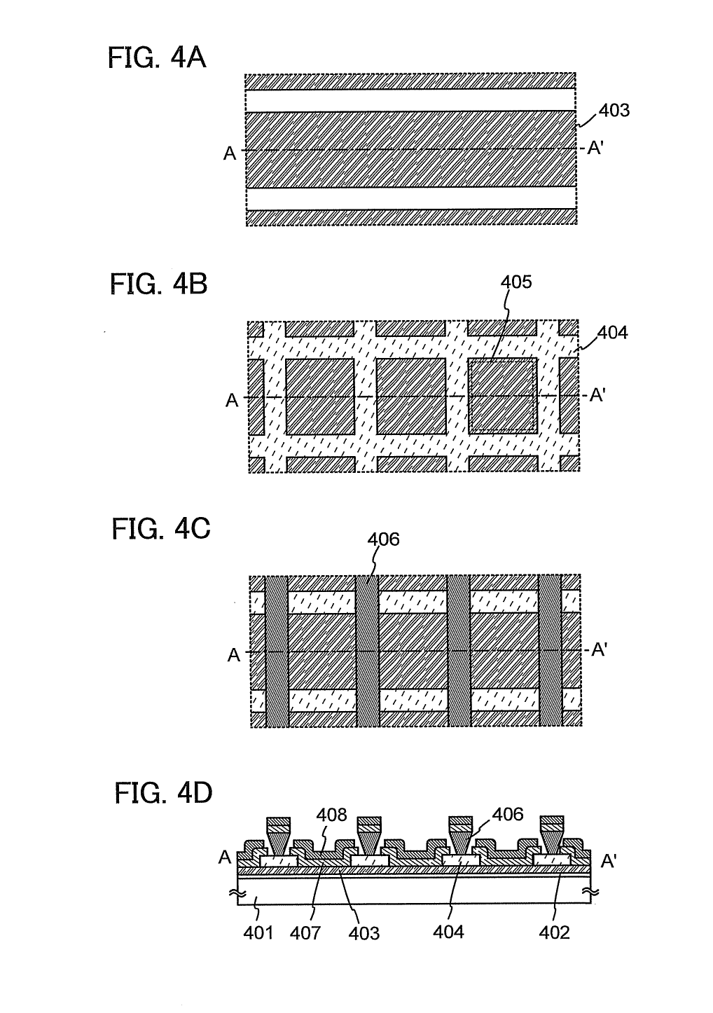

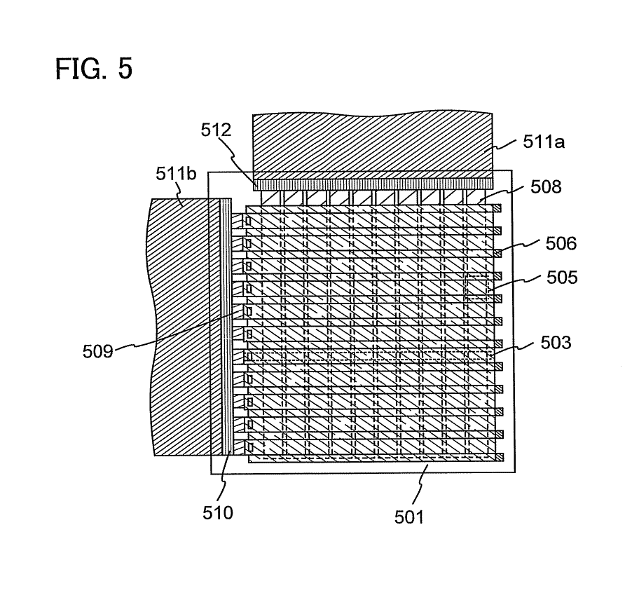

FIGS. 4A to 4D and FIG. 5 exemplify passive-matrix light-emitting devices.

In a passive-matrix (also called simple-matrix) light-emitting device, a plurality of anodes arranged in stripes (in stripe form) is provided orthogonal to a plurality of cathodes arranged in stripes. A light-emitting layer is formed at each intersection. Therefore, light is emitted from a light-emitting layer (hereinafter, referred to as a pixel) at an intersection of an anode selected (to which a voltage is applied) and a cathode selected.

FIGS. 4A to 4C are top views of a pixel portion before sealing. FIG. 4D is a cross-sectional view taken along dashed line A-A′ in each of FIGS. 4A to 4C.

Over a substrate 401, an insulating layer 402 is formed as a base insulating layer. Note that the base insulating layer is not a necessary component and thus formed as needed. A plurality of first electrodes 403 is arranged at regular intervals over the insulating layer 402 (see FIG. 4A).

In addition, a partition 404 having openings in regions corresponding to pixels is provided over the first electrodes 403. The partition 404 having openings is formed using an organic material (polyimide, acrylic, polyimide, polyimide amide, resist, or benzocyclobutene), an inorganic material (e.g., a SiOₓ film including an alkyl group), or the like. Note that openings 405 corresponding to the pixels serve as light-emitting regions (see FIG. 4B).

Over the partition 404, a plurality of partitions 406 is provided so as to intersect with the first electrodes 403 (see FIG. 4C). The partitions 406 are each reversely tapered and arranged in parallel to one another.

In regions over the first electrodes 403 where the partitions 406 are not formed, EL layers 407 and second electrodes 408 are provided in that order (see FIG. 4D). Here, the EL layers 407 and the second electrodes 408 are formed as plural portions, which are electrically isolated from each other. Such a structure can be obtained by forming the partitions 406 the height of which exceeds the sum of the thicknesses of the EL layers 407 and the second electrodes 408.

The second electrodes 408 extend in the direction in which they intersect with the first electrodes 403. Note that over the partitions 406, a layer of the same material as the EL layer 407 and a layer of the same material as the second electrode 408 are also formed, which are isolated from the EL layer 407 and the second electrode 408.

Note that the first electrode 403 and the second electrode 408 may serve as an anode and a cathode, respectively, or vice versa. The stack structure of the EL layer 407 is adjusted depending on the polarity of the electrodes, as appropriate.

Further, the substrate 401 may be sealed so that a light-emitting element is provided in a sealed space. Sealing is carried out with an adhesive such as a seal material to attach the substrate 401 to a sealing can or a sealant. Such sealing can suppress deterioration of the light-emitting element. Note that the sealed space may be filled with filler, a dried inert gas, a drying agent (a desiccant), or the like. Sealing a drying agent enables removal of a minute amount of moisture, whereby deterioration of the light-emitting element which is caused by moisture is suppressed. Note that as a drying agent, a substance that adsorbs moisture by chemical adsorption can be used. For example, oxides of alkaline earth metals such as calcium oxide and barium oxide can be used. Alternatively, a substance that adsorbs moisture by physical adsorption, such as zeolite or silicagel, may be used.

Next, FIG. 5 illustrates a structure of a passive-matrix light-emitting device as illustrated in FIGS. 4A to 4D, on which an FPC and the like are mounted.

In a pixel portion formed over a substrate 501 in FIG. 5, scan lines and data lines are arranged to intersect with each other so that they are orthogonal to each other. Note that the first electrodes 403 in FIGS. 4A to 4D correspond to scan lines 503 in FIG. 5, the second electrodes 408 in FIGS. 4A to 4D correspond to data lines 508 in FIG. 5, and the partitions 406 in FIGS. 4A to 4D correspond to partitions 506 in FIG. 5. An EL layer is formed between the data line 508 and the scan line 503, and a region 505 corresponds to one pixel.

Note that the scan lines 503 are electrically connected at their ends to connection wirings 509, and the connection wirings 509 are connected to an FPC 511b through an input terminal 510. The data lines 508 are connected to an FPC 511a through an input terminal 512.

For example, a surface where light is extracted may be provided with an optical film such as a polarizing plate, a circularly polarizing plate (including an elliptically polarizing plate), a retardation plate (a λ/4 plate or a λ/2 plate), a color filter, or an anti-reflection film. In addition, the surface where light is extracted or a surface of the various films may be subjected to treatment. For example, by forming a slightly uneven surface, reflected light diffuses to reduce glare.

Note that although FIG. 5 illustrates the example in which an IC chip including a driver circuit is not provided over the substrate, an IC chip may be mounted on the substrate. As a method for mounting an IC chip, a COG method, a wire bonding method, TCP, or the like can be used.

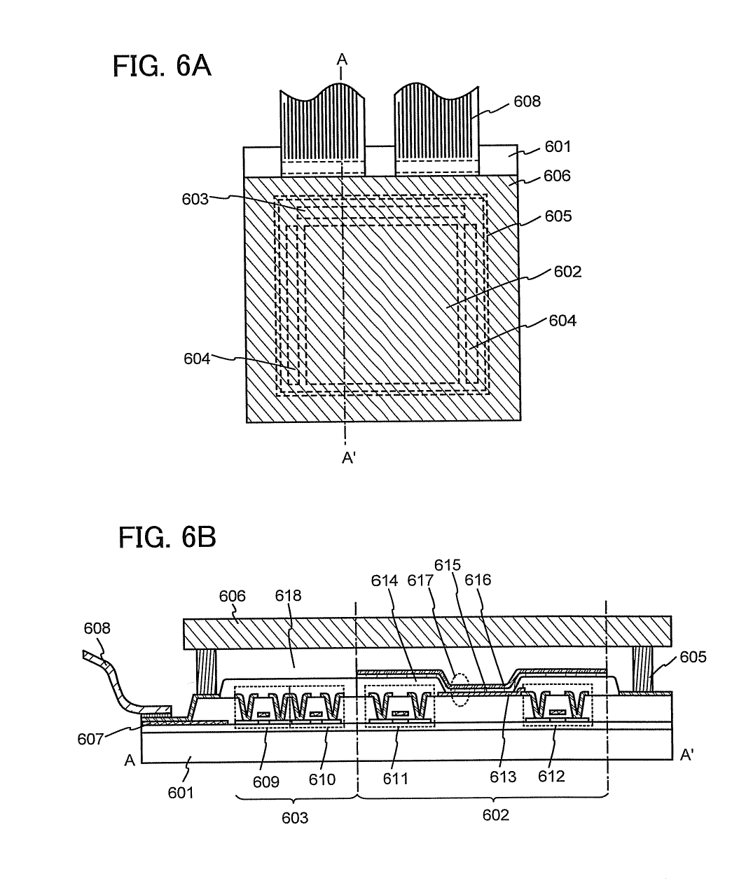

FIGS. 6A and 6B illustrate an example of an active matrix light-emitting device.

FIG. 6A is a top view of the light-emitting device. FIG. 6B is a cross-sectional view taken along dashed line A-A′ in FIG. 6A.

The active matrix light-emitting device of this embodiment includes a pixel portion 602, a driver circuit portion 603 (a source side driver circuit), and a driver circuit portion 604 (a gate side driver circuit) which are provided over an element substrate 601. The pixel portion 602, the driver circuit portion 603, and the driver circuit portion 604 are sealed with a sealant 605 between the element substrate 601 and a sealing substrate 606 (see FIG. 6A).

In addition, over the element substrate 601, a lead wiring 607 for connecting an external input terminal is provided. Note that here, an example is described in which a flexible printed circuit (FPC) is provided as the external input terminal. Although only the FPC 608 is illustrated in FIGS. 6A and 6B, this FPC may be provided with a printed wiring board (PWB). The term light-emitting device in this specification and the like includes not only a light-emitting device body but also a light-emitting device to which an FPC, a PWB, or the like is attached.

In the driver circuit portion 603, a CMOS circuit is formed by combining an n-channel TFT 609 and a p-channel TFT 610 (see FIG. 6B). It is needless to say that the circuit configuration is not limited to this example, and any of various circuits such as CMOS circuits, PMOS circuits, or NMOS circuits can be applied. In addition, although a driver circuit-integrated type where the driver circuit is formed over the substrate is described in this embodiment, the present invention is not to be construed as being limited to this structure. The driver circuit can be formed outside. Note that FIG. 6B exemplifies just the driver circuit portion 603 which is the source side driver circuit and the pixel portion 602.

The pixel portion 602 has plural pixels, each of which includes a switching TFT 611, a current control TFT 612, and an anode 613 which is electrically connected to an electrode (a source or drain electrode) of the current control TFT 612. Note that an insulator 614 is formed to cover the edge portion of the anode 613. Further, for the insulator 614, either a negative photosensitive material which becomes insoluble in an etchant by light or a positive photosensitive material which becomes soluble in an etchant by light can be used. Without limitation to an organic compound, an inorganic compound such as silicon oxide or silicon oxynitride can be used.

Preferably, an upper edge portion or a lower edge portion of the insulator 614 is a curved surface having a specific curvature radius. The curved surface contributes to improvement of coverage by a film which is to be formed over the insulator 614. For example, when a positive photosensitive acrylic resin is used as a material for the insulator 614, the upper edge portion thereof is preferably formed as a curved surface having a curvature radius of 0.2 to 3 μm.

Over the anode 613, an EL layer 615 and a cathode 616 are stacked. Here, by applying an ITO film to the anode 613 and applying a stack of a titanium nitride film and a film including aluminum as the main component or of a titanium nitride film, a film including aluminum as the main component, and a titanium nitride film to a wiring of the current control TFT 612 which is connected to the anode 613, favorable ohmic contact with the ITO film can be obtained and resistance of the wiring can be kept low. Note that although not illustrated here, the cathode 616 is electrically connected to the FPC 608 which is an external input terminal.

Note that in the EL layer 615, at least a light-emitting layer is provided, and in addition to the light-emitting layer, a hole-injection layer, a hole-transport layer, an electron-transport layer, an electron-injection layer, and/or the like may be provided. The anode 613, the EL layer 615, and the cathode 616 are stacked to form a light-emitting element 617.

In addition, although the cross-section in FIG. 6B illustrates one light-emitting element 617, a plurality of light-emitting elements is arranged in matrix in the pixel portion 602. Further, full-color display can be achieved by providing light-emitting elements that emit light of three colors (R, G, and B) as selected in the pixel portion 602. Color filters may be used in combination to perform full-color display.

The light-emitting element 617 is provided in a space 618 surrounded by the element substrate 601, the sealing substrate 606, and the sealant 605. Note that the space 618 may be filled with an inert gas (nitrogen, argon, or the like) or any other material such as the sealant 605.

As a material for the sealant 605, an epoxy resin is preferably used. It is desirable to use a material that allows permeation of moisture or oxygen as little as possible. As a material for the element substrate 601 or the sealing substrate 606, a plastic substrate formed of fiberglass-reinforced plastics (FRP), polyvinyl fluoride (PVF), polyester, acrylic, or the like can be used besides a glass substrate or a quartz substrate.

Note that this embodiment can be used in appropriate combination with any structure described in the above embodiment.

### Embodiment 6

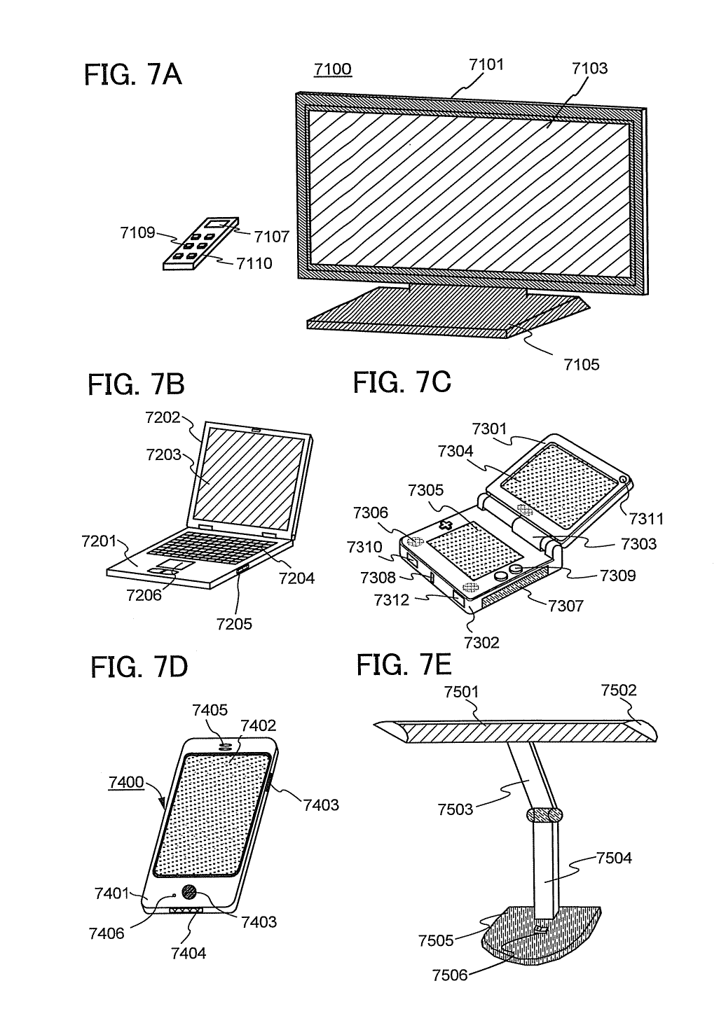

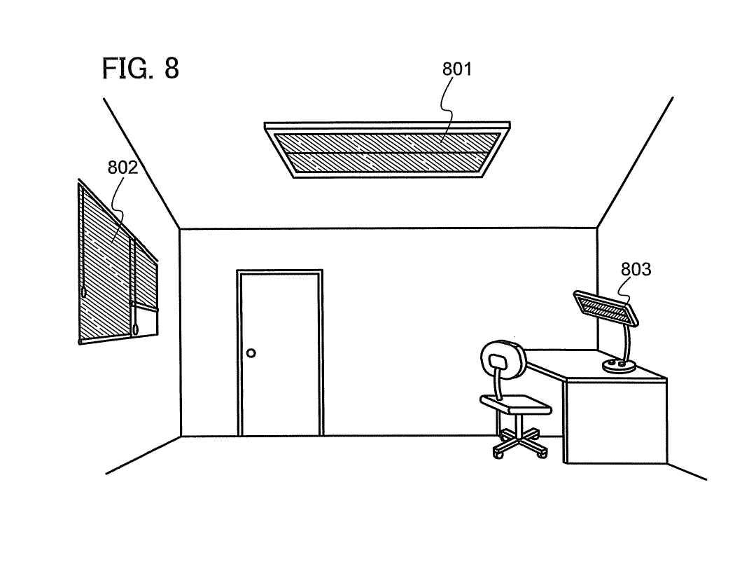

In this embodiment, with reference to FIGS. 7A to 7E and FIG. 8, description is given of examples of a variety of electronic devices and lighting devices that are completed by using any light-emitting device which is one embodiment of the present invention.

Examples of the electronic devices to which the light-emitting device is applied include television sets (also referred to as televisions or television receivers), monitors of computers or the like, cameras such as digital cameras or digital video cameras, digital photo frames, cellular phones (also referred to as cellular phones or cellular phone sets), portable game consoles, portable information terminals, audio reproducing devices, large-sized game machines such as pachinko machines, and the like. Some specific examples of these electronic devices and a lighting device are illustrated in FIGS. 7A to 7E.

FIG. 7A illustrates an example of a television set 7100. In the television set 7100, a display portion 7103 is incorporated in a housing 7101. Images can be displayed by the display portion 7103, in which the light-emitting device can be used. Here, the housing 7101 is supported by a stand 7105.

The television set 7100 can be operated with an operation switch of the housing 7101 or a separate remote controller 7110. Channels and volume can be controlled with an operation key 7109 of the remote controller 7110 so that an image displayed on the display portion 7103 can be controlled. Furthermore, the remote controller 7110 may be provided with a display portion 7107 for displaying data output from the remote controller 7110.

Note that the television set 7100 is provided with a receiver, a modem, and the like. With the use of the receiver, general television broadcasting can be received. Moreover, when the television set is connected to a communication network with or without wires via the modem, one-way (from a sender to a receiver) or two-way (between a sender and a receiver or between receivers) information communication can be performed.

FIG. 7B illustrates an example of a computer. This computer includes a main body 7201, a housing 7202, a display portion 7203, a keyboard 7204, an external connecting port 7205, a pointing device 7206, and the like. Note that the computer is manufactured by using the light-emitting device for the display portion 7203.

FIG. 7C illustrates an example of a portable amusement machine. This portable amusement machine includes two housings: a housing 7301 and a housing 7302. The housings 7301 and 7302 are connected with a connection portion 7303 so as to be opened and closed. A display portion 7304 and a display portion 7305 are incorporated in the housing 7301 and the housing 7302, respectively. In addition, the portable amusement machine illustrated in FIG. 7C includes a speaker portion 7306, a recording medium insertion portion 7307, an LED lamp 7308, an input means (an operation key 7309, a connection terminal 7310, a sensor 7311 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), or a microphone 7312), and the like. It is needless to say that the structure of the portable amusement machine is not limited to the above as long as the light-emitting device is used for at least either the display portion 7304 or the display portion 7305, or both. The portable amusement machine may include other accessory equipment as appropriate. The portable amusement machine illustrated in FIG. 7C has a function of reading a program or data stored in a recording medium to display it on the display portion, and a function of sharing information with another portable amusement machine by wireless communication. The portable amusement machine illustrated in FIG. 7C can have any other various functions without limitation to the above.

FIG. 7D illustrates an example of a cellular phone. The cellular phone 7400 is provided with a display portion 7402 incorporated in a housing 7401, operation buttons 7403, an external connection port 7404, a speaker 7405, a microphone 7406, and the like. Note that the light-emitting device is used for the display portion 7402 of the cellular phone 7400.

When the display portion 7402 of the cellular phone 7400 illustrated in FIG. 7D is touched with a finger or the like, data can be input into the cellular phone 7400. Furthermore, operations such as making calls and composing mails can be performed by touching the display portion 7402 with a finger or the like.

There are mainly three screen modes for the display portion 7402. The first mode is a display mode mainly for displaying images. The second mode is an input mode mainly for inputting data such as text. The third mode is a display-and-input mode in which two modes of the display mode and the input mode are combined.

For example, for operations such as making calls and composing mails, the display portion 7402 is set to a text input mode (second mode) mainly for inputting text so that text can be input. In this case, a keyboard or number buttons are preferably displayed on the display portion 7402.

By providing a detection device which includes a sensor for detecting inclination, such as a gyroscope or an acceleration sensor, inside the cellular phone 7400, the direction of the cellular phone 7400 is determined so that display on the screen of the display portion 7402 can be automatically switched.

In addition, the screen mode is switched by, for example, touching the display portion 7402 or operating the operation buttons 7403 of the housing 7401. Alternatively, the screen mode may be switched depending on the kind of images displayed on the display portion 7402. For example, when a signal of an image displayed on the display portion is of moving image data, the screen mode is switched to the display mode (first mode). When the signal is of text data, the screen mode is switched to the input mode (second mode).

Furthermore, when input by touching the display portion 7402 is not performed for a certain period, a controlling operation may be performed: for example, the screen mode may be switched from the input mode (first mode) to the display mode (second mode).

The display portion 7402 may function as an image sensor. For example, an image of a palm print, a fingerprint, or the like is taken by touching the display portion 7402 with the palm or the finger, whereby personal authentication can be performed. Furthermore, by providing a backlight or a sensing light source emitting a near-infrared light for the display portion, an image of a finger vein, a palm vein, or the like can also be taken.

FIG. 7E illustrates a desk lamp including a lighting portion 7501, a shade 7502, an adjustable arm 7503, a support 7504, a base 7505, and a power supply 7506. The desk lamp is manufactured using the light-emitting device in the lighting portion 7501. Note that the term lighting device also includes ceiling lights, wall lights, and the like.

FIG. 8 illustrates an example in which the light-emitting device is used for an indoor lighting device 801. The light-emitting device enables an increase in emission area, and therefore can be used as a large-sized lighting device. Furthermore, the light-emitting device may be used as a lighting device 802 which can be rolled up. In addition, a desk lamp 803 illustrated in FIG. 7E may be used together in the room provided with the interior lighting device 801.

The electronic devices, lighting devices, and the like as illustrated above can be provided by application of the light-emitting device described in the above embodiment, for example. Thus, the applicable range of the light-emitting device is wide so that the light-emitting device can be applied to electronic devices in a variety of fields.

Note that the structure described in this embodiment can be combined with a structure described in any of the above embodiments, as appropriate.

### Example 1

In this example, a method for synthesizing the oxadiazole derivative (or carbazole derivative) represented by Structural Formula (100), 3-phenyl-9-[4-(5-phenyl-1,3,4-oxadiazol-2-yl)phenyl]-9H-carbazole (abbreviation: CO11II), will be specifically described.

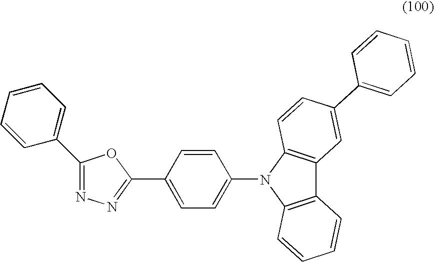

The synthetic scheme of 3-phenyl-9-[4-(5-phenyl-1,3,4-oxadiazol-2-yl)phenyl]-9H-carbazole is shown in (B-1).

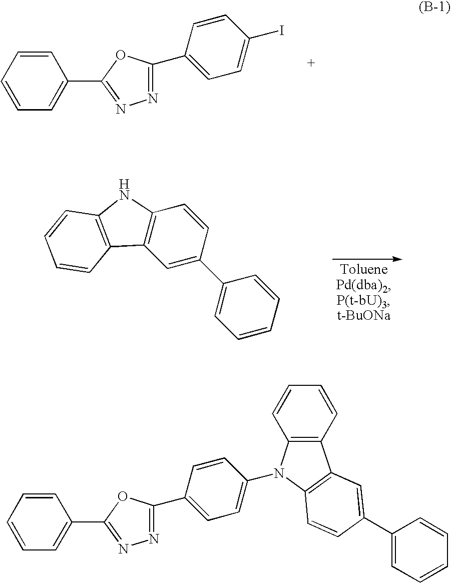

In a 100 mL three-necked flask were placed 2.3 g (6.6 mmol) of 2-(4-iodophenyl)-5-phenyl-1,3,4-oxadiazole, 1.6 g (6.6 mmol) of 3-phenyl-9H-carbazole, and 1.4 g (15 mmol) of sodium tert-butoxide, and the atmosphere in the flask was replaced with nitrogen. To this mixture were added 30 mL of toluene and 0.2 mL of a 10% hexane solution of tri(tert-butyl)phosphine. The pressure in the flask was reduced with an aspirator to degas this mixture, and then the atmosphere in the flask was replaced with nitrogen. To this mixture was added 0.058 g (0.10 mmol) of bis(dibenzylideneacetone)palladium(0). The resulting mixture was stirred under a nitrogen stream at 80° C. for 15 hours. After that, toluene was added to this mixture, and this suspension was washed with a saturated aqueous solution of sodium carbonate and brine in this order. Then, the organic layer was dried by addition of magnesium sulfate. After that, this mixture was suction filtered. The resulting filtrate was suction filtered through Celite (Wako Pure Chemical Industries. Ltd., Catalog No. 531-16855). The resulting filtrate was concentrated to give a compound, which was purified by silica gel column chromatography. The column chromatography was performed first using toluene as a developing solvent and then using a mixed solvent of a 4:1 ratio of toluene to ethyl acetate as a developing solvent. The fractions obtained were concentrated to give a solid. Acetone was added to this solid, followed by ultrasonic cleaning. This mixture was subjected to suction filtration to collect a solid. The collected solid was recrystallized with a mixed solvent of chloroform and hexane, so that 2.0 g of a white powdered solid was obtained in 64% yield.

By a train sublimation method, 1.1 g of the obtained white solid was purified. Under a reduced pressure of 3.0 Pa with a flow rate of argon at 5 mL/min, the sublimation purification was carried out at 240° C. for 16 hours. The amount of the compound was 0.98 g, and the yield thereof was 89%.

By a nuclear magnetic resonance (NMR) method, this compound was confirmed to be 3-phenyl-9-[4-(5-phenyl-1,3,4-oxadiazol-2-yl)phenyl]-9H-carbazole (abbreviation: CO11II).

The following are data of the ¹H NMR of the obtained compound: ¹H NMR (CDCl₃, 300 MHz): δ=7.30-7.76 (m, 13H), 7.79 (d, J=8.3 Hz, 2H), 8.14-8.24 (m, 3H), 8.35 (sd, J=1.5 Hz, 1H), 8.39 (d, J=8.8 Hz, 2H)

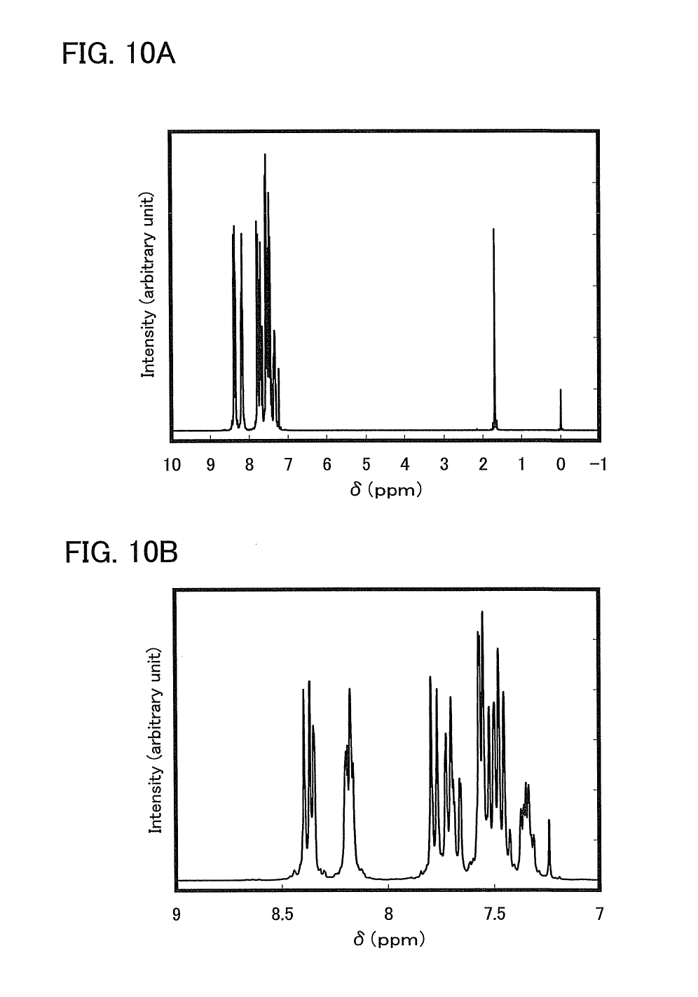

In addition, FIGS. 10A and 10B show ¹H NMR charts. Note that FIG. 10B is a chart showing an enlarged part in the range of 7.0 ppm to 9.0 ppm in FIG. 10A.

Further, the glass transition temperature of CO11II was measured with a differential scanning calorimeter (Pyris 1 DSC, manufactured by Perkin Elmer Co., Ltd.) and found to be 88° C. These results indicate that CO11II is a highly heat-resistant material.

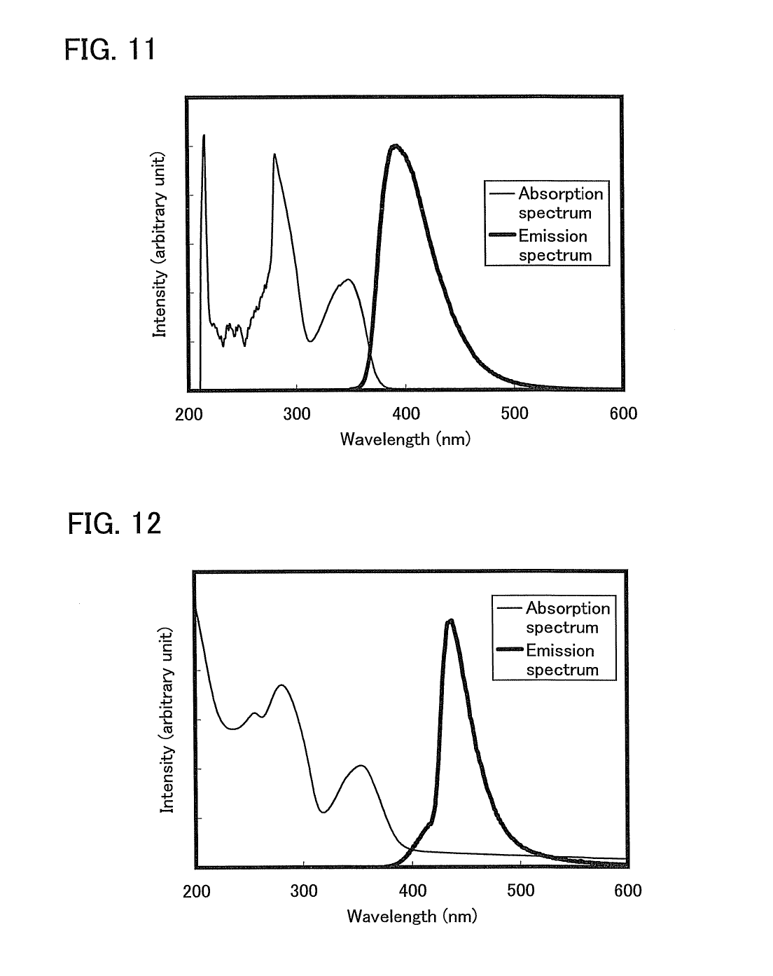

FIG. 11 shows an absorption spectrum and an emission spectrum of a toluene solution of CO11II, and FIG. 12 shows an absorption spectrum and an emission spectrum of a thin film of CO11II. An ultraviolet-visible spectrophotometer (V-550, manufactured by JASCO Corporation) was used for the measurements. To prepare samples, the solution was put to a quartz cell while the thin film was obtained by evaporation onto a quartz substrate. The absorption spectrum of the solution was obtained by subtracting that of a quartz cell containing only toluene, which is shown in FIG. 11. The absorption spectrum of the thin film was obtained by subtracting that of the quartz substrate, which is shown in FIG. 12. In FIG. 11 and FIG. 12, the horizontal axis represents wavelength (nm) and the vertical axis represents intensity (arbitrary unit). In the case of the toluene solution, absorption was observed at about 347 nm, and a maximum emission wavelength of the solution was 393 nm (excitation wavelength: 349 nm). Further, in the case of the thin film, absorption was observed at about 353 nm, and a maximum emission wavelength of the solution was 437 nm (excitation wavelength: 359 nm).

Furthermore, the HOMO level and LUMO level of a thin film of CO11II were determined. The value of the HOMO level was obtained by converting the value of the ionization potential measured with a photoelectron spectrometer (AC-2, manufactured by Riken Keiki Co., Ltd.) into a negative value. In addition, the value of the LUMO level was obtained in such a manner that the absorption edge was obtained from Tauc plot, with an assumption of direct transition, using data on the absorption spectrum of the thin film of CO11II which was shown in FIG. 12, and added as an optical energy gap to the value of the HOMO level. The results reveal that the HOMO level, energy gap, and LUMO level of CO11II are −5.71 eV, 3.26 eV, and −2.45 eV, respectively.

Thus, CO11II is found to have a large energy gap.

In addition, the oxidation-reduction characteristics of CO11II were measured. Cyclic voltammetry (CV) measurement was here employed. Further, an electrochemical analyzer (ALS model 600A, a product of BAS Inc.) was used for the measurements.

For a solution used in the CV measurements, dehydrated dimethylformamide (DMF, produced by Sigma-Aldrich Inc., 99.8%, Catalog No. 22705-6) was used as a solvent. Tetra-n-butylammonium perchlorate (n-Bu₄NClO₄, produced by Tokyo Chemical Industry Co., Ltd., Catalog No. T0836), which was a supporting electrolyte, was dissolved in the solvent such that the concentration of tetra-n-butylammonium perchlorate was 100 mmol/L. Furthermore, the substance that was to be measured was dissolved in the solution such that the concentration thereof was 1 mmol/L. In addition, a platinum electrode (PTE platinum electrode, produced by BAS Inc.) was used as a working electrode, a platinum electrode (Pt counter electrode for VC-3, (5 cm), produced by BAS Inc.) was used as an auxiliary electrode, and an Ag/Ag⁺ electrode (RE-7 reference electrode for nonaqueous solvent, produced by BAS Inc.,) was used as a reference electrode. Note that the measurements were conducted at room temperature.

The oxidation characteristics of CO11II were examined by 100 cycles of measurements in which a scan for changing the potential of the working electrode with respect to the reference electrode from −0.155 V to 1.05 V and then from 1.05 V to −0.155 V was set to one cycle. Note that the scan rate for the CV measurements was set to 0.1 V/s.

The reduction characteristics of CO11II were examined by 100 cycles of measurements in which a scan for changing the potential of the working electrode with respect to the reference electrode from −1.31 V to −2.45 V and then from −2.45 V to −1.31 V was set to one cycle. Note that the scan rate for the CV measurements was set to 0.1 V/s.

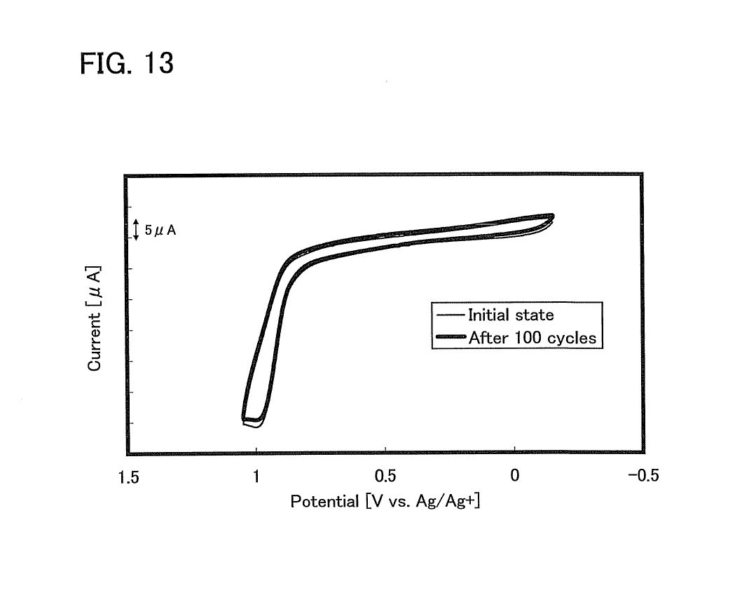

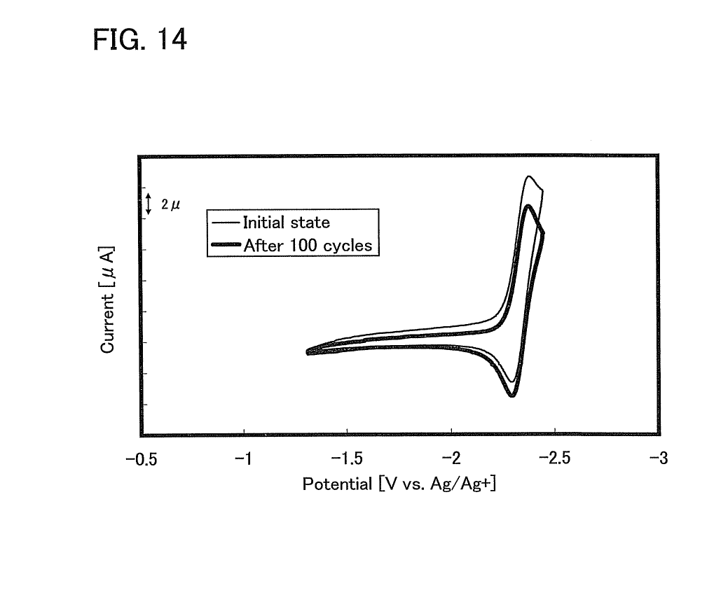

FIG. 13 shows CV measurement results of the oxidation characteristics of CO11II, and FIG. 14 shows CV measurement results of the reduction characteristics of CO11II. In FIG. 13 and FIG. 14, the horizontal axis represents potential (V) of the working electrode with respect to the reference electrode, and the vertical axis represents value of a current (μA) flowing between the working electrode and the auxiliary electrode. As shown in FIG. 13, a current exhibiting oxidation is observed at about +0.93 V (vs. the Ag/Ag⁺ electrode). As shown in FIG. 14, a current exhibiting reduction is observed at about −2.34 V (vs. the Ag/Ag⁺ electrode).

After the 100 cycles of measurements in which the scan was repeated, there was no significant change in the peak position of the CV curves exhibiting the oxidation and reduction reactions. Accordingly, it is found that the oxadiazole derivative that is one embodiment of the disclosed invention is stable to repetitive oxidation-reduction reactions.

In addition, the optimal molecular structure of CO11II in the ground state was calculated using the density functional theory (DFT). In the DFT, the total energy is represented as the sum of potential energy, electrostatic energy between electrons, electronic kinetic energy, and exchange-correlation energy including all the complicated interactions between electrons. Also in the DFT, an exchange-correlation interaction approximates a functional (a function of another function) of one electron potential expressed as electron density to enable highly accurate calculations. Here, B3LYP which was a hybrid functional was used to specify the weight of each parameter related to exchange-correlation energy. In addition, as a basis function, 6-311 (a basis function of a triple-split valence basis set using three contraction functions for each valence orbital) was applied to all the atoms. By the above basis function, for example, orbits of is to 3s are considered in the case of hydrogen atoms while orbits of is to 4s and 2p to 4p are considered in the case of carbon atoms. Furthermore, to improve calculation accuracy, the p function and the d function as polarization basis sets were added respectively to hydrogen atoms and atoms other than hydrogen atoms.

Note that Gaussian 03 was used as a quantum chemistry computational program. A high performance computer (manufactured by SGI Japan, Ltd., Altix 4700) was used for the calculations.

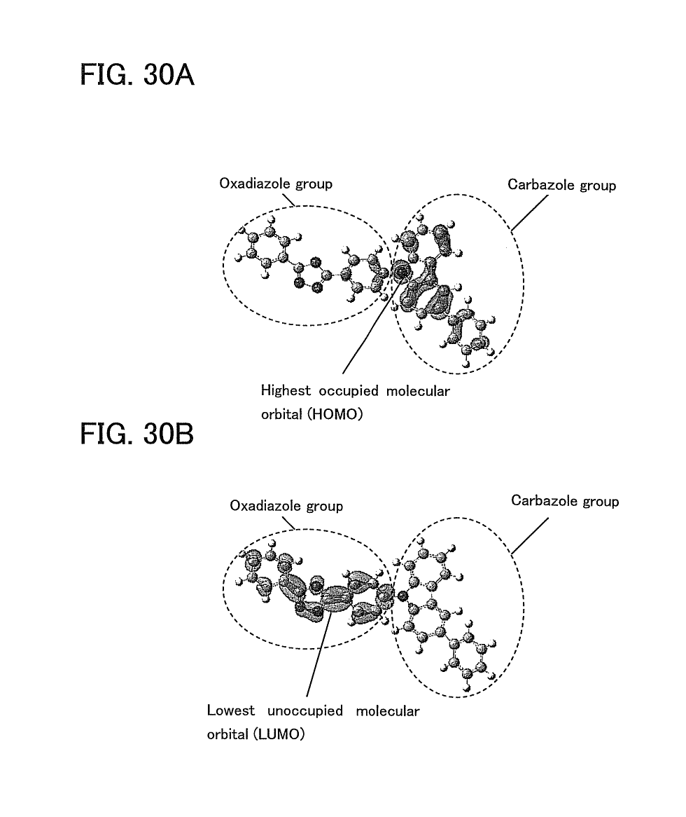

FIGS. 30A and 30B show respectively the highest occupied molecular orbital (HOMO) and lowest unoccupied molecular orbital (LUMO) of CO11II, which were found by the calculations. FIG. 30A shows the highest occupied molecular orbital (HOMO), and FIG. 30B shows the lowest unoccupied molecular orbital (LUMO). In the drawings, the spheres represent atoms forming CO11II and cloud-like objects around atoms represent orbits that are subjected to the calculations. Note that FIGS. 30A and 30B are visualization views of calculation results of the optimal molecular structures obtained by Gaussview 4.1, which is software visualizing computational results.

FIGS. 30A and 30B reveal that the unoccupied molecular orbital and lowest unoccupied molecular orbital of CO11II exist in a carbazole group and an oxadiazole group, respectively. In other words, the carbazole group contributes to the hole-transport property of CO11II while the oxadiazole group contributes to the electron-transport property thereof, which proves the high bipolar character of CO11II.

### Example 2

In this example, a method for synthesizing the oxadiazole derivative (or carbazole derivative) represented by Structural Formula (200), 3,6-diphenyl-9-[4-(5-phenyl-1,3,4-oxadiazol-2-yl)phenyl]-9H-carbazole (abbreviation: CO11III), will be specifically described.

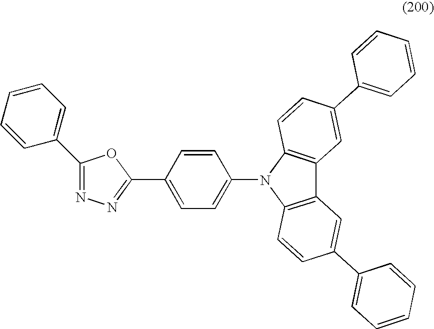

The synthetic scheme of 3,6-diphenyl-9-[4-(5-phenyl-1,3,4-oxadiazol-2-yl)phenyl]-9H-carbazole is shown in (C-1).

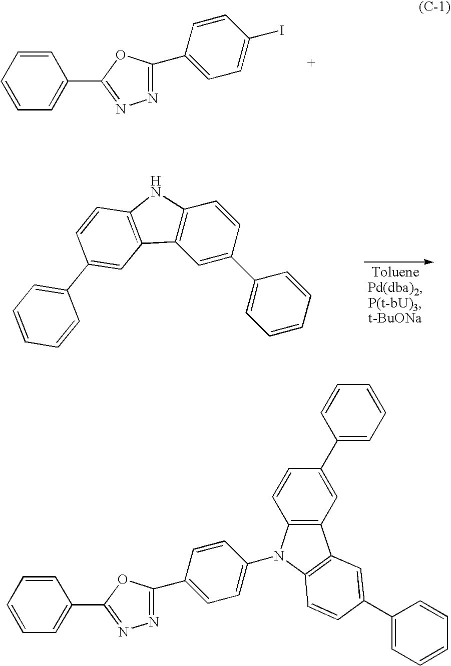

In a 100 mL three-necked flask were placed 1.0 g (2.9 mmol) of 2-(4-iodophenyl)-5-phenyl-1,3,4-oxadiazole, 0.92 g (2.9 mmol) of 3,6-diphenyl-9H-carbazole, and 0.61 g (6.3 mmol) of sodium tert-butoxide, and the atmosphere in the flask was replaced with nitrogen. To this mixture were added 15 mL of toluene and 0.10 mL of a 10% hexane solution of tri(tert-butyl)phosphine. The pressure in the flask was reduced with an aspirator to degas this mixture, and then the atmosphere in the flask was replaced with nitrogen. To this mixture was added 0.025 g (0.043 mmol) of bis(dibenzylideneacetone)palladium(0). The resulting mixture was stirred under a nitrogen stream at 110° C. for 10 hours. After that, toluene was added to this mixture, and the organic layer was washed with a saturated aqueous solution of sodium carbonate and brine in this order. Then, the organic layer was dried by addition of magnesium sulfate. After that, this mixture was suction filtered. The resulting filtrate was concentrated to give a compound, which was purified by silica gel column chromatography. The column chromatography was performed first using toluene as a developing solvent and then using a mixed solvent of a 8:1 ratio of toluene to ethyl acetate as a developing solvent. The fractions obtained were concentrated to give a solid. Acetone was added to this solid, followed by ultrasonic cleaning. This mixture was subjected to suction filtration to collect a solid. The solid collected was recrystallized with a mixed solvent of chloroform and methanol to give 0.80 g of a white powdered solid in 52% yield.

By a train sublimation method, 0.80 g of the obtained white solid was purified. Under a reduced pressure of 2.7 Pa with a flow rate of argon at 5 mL/min, the sublimation purification was carried out at 260° C. for 15 hours. The amount of the compound was 0.61 g, and the yield thereof was 76%.

By a nuclear magnetic resonance (NMR) method, this compound was confirmed to be 3,6-diphenyl-9-[4-(5-phenyl-1,3,4-oxadiazol-2-yl)phenyl]-9H-carbazole (abbreviation: CO11III).

The following are data of the ¹H NMR of the obtained compound: ¹H NMR (CDCl₃, 300 MHz): δ=7.29-7.83 (m, 19H), 8.13-8.21 (m, 2H), 8.35-8.41 (m, 4H)

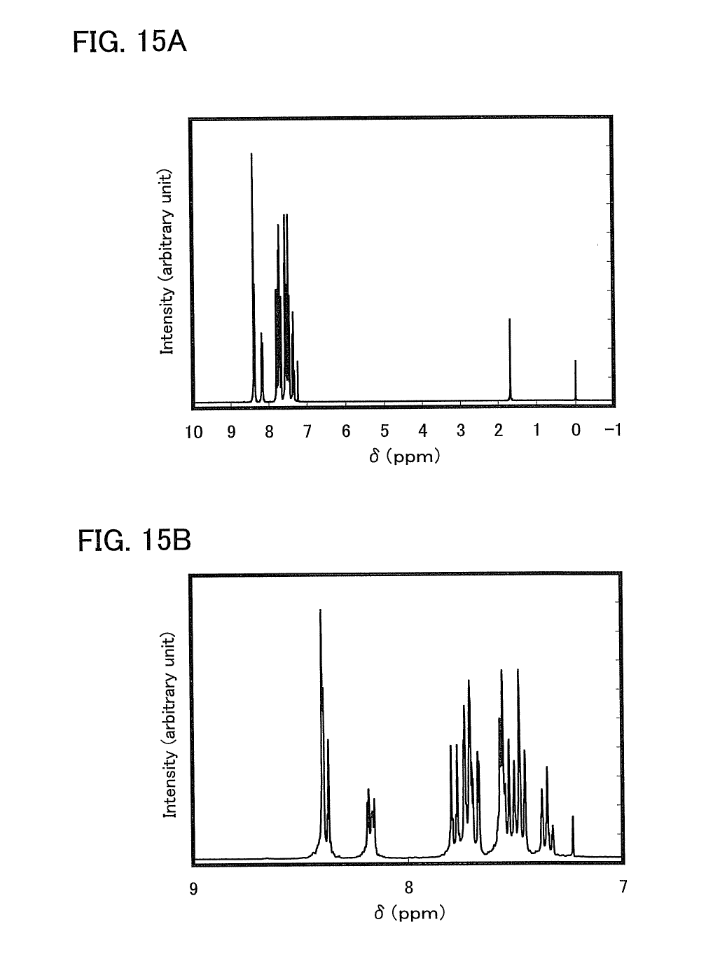

In addition, FIGS. 15A and 15B show ¹H NMR charts. Note that FIG. 15B is a chart showing an enlarged part in the range of 7.0 ppm to 9.0 ppm in FIG. 15A.

Further, the glass transition temperature of CO11III was measured with a differential scanning calorimeter (Pyris 1 DSC, manufactured by Perkin Elmer Co., Ltd.) and found to be 114° C. These results indicate that CO11III is a highly heat-resistant material.

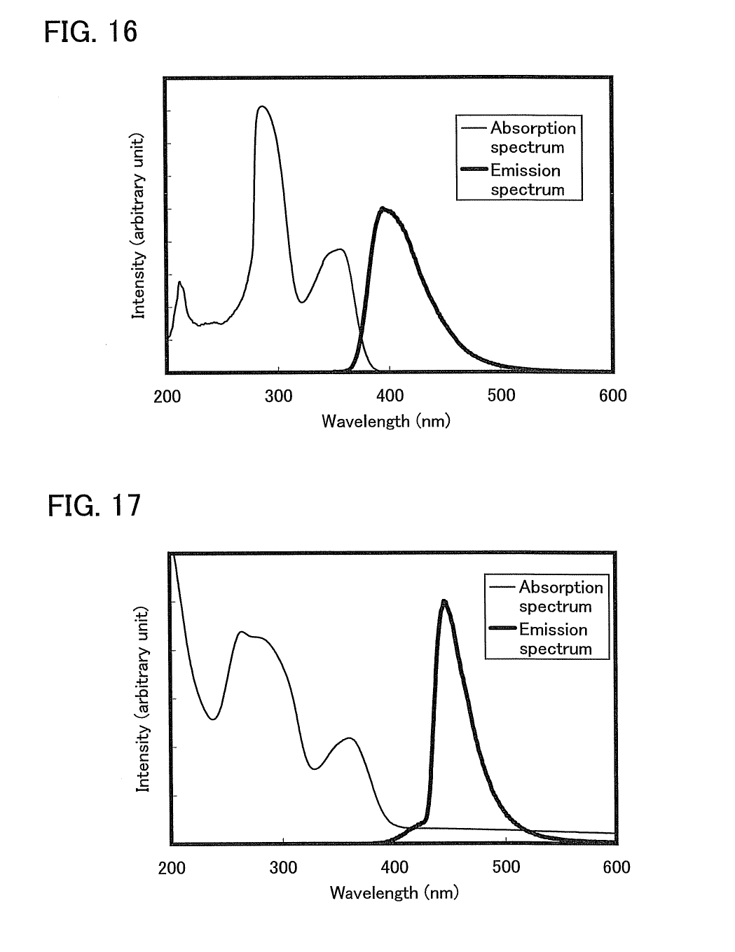

FIG. 16 shows an absorption spectrum and an emission spectrum of a toluene solution of CO11III, and FIG. 17 shows an absorption spectrum and an emission spectrum of a thin film of CO11III. An ultraviolet-visible spectrophotometer (V-550, manufactured by JASCO Corporation) was used for the measurements. To prepare samples, the solution was put to a quartz cell while the thin film was obtained by evaporation onto a quartz substrate. The absorption spectrum of the solution was obtained by subtracting that of a quartz cell containing only toluene, which is shown in FIG. 16. The absorption spectrum of the thin film was obtained by subtracting the absorption spectrum of the quartz substrate, which is shown in FIG. 17. In FIG. 16 and FIG. 17, the horizontal axis represents wavelength (nm) and the vertical axis represents intensity (arbitrary unit). In the case of the toluene solution, absorption was observed at about 351 nm, and a maximum emission wavelength of the solution was 395 nm (excitation wavelength: 351 nm). Further, in the case of the thin film, absorption was observed at about 360 nm, and a maximum emission wavelength of the solution was 447 nm (excitation wavelength: 366 nm).

Furthermore, the HOMO level and LUMO level of a thin film of CO11II were determined. The value of the HOMO level was obtained by converting the value of the ionization potential measured with a photoelectron spectrometer (AC-2, manufactured by Riken Keiki Co., Ltd.) into a negative value. In addition, the value of the LUMO level was obtained in such a manner that the absorption edge was obtained from Tauc plot, with an assumption of direct transition, using data on the absorption spectrum of the thin film of CO11II which was shown in FIG. 17, and added as an optical energy gap to the value of the HOMO level. The results reveal that the HOMO level, energy gap, and LUMO level of CO11III are −5.67 eV, 3.21 eV, and −2.46 eV, respectively.

Thus, CO11III is found to have a large energy gap.

In addition, the oxidation-reduction characteristics of CO11II were measured. Cyclic voltammetry (CV) measurement was here employed. Further, an electrochemical analyzer (ALS model 600A, a product of BAS Inc.) was used for the measurements.

For a solution used in the CV measurements, dehydrated dimethylformamide (DMF, produced by Sigma-Aldrich Inc., 99.8%, Catalog No. 22705-6) was used as a solvent. Tetra-n-butylammonium perchlorate (n-Bu₄NClO₄, produced by Tokyo Chemical Industry Co., Ltd., Catalog No. T0836), which was a supporting electrolyte, was dissolved in the solvent such that the concentration of tetra-n-butylammonium perchlorate was 100 mmol/L. Furthermore, the substance that was to be measured was dissolved in the solution such that the concentration thereof was 1 mmol/L. In addition, a platinum electrode (PTE platinum electrode, produced by BAS Inc.) was used as a working electrode, a platinum electrode (Pt counter electrode for VC-3, (5 cm), produced by BAS Inc.) was used as an auxiliary electrode, and an Ag/Ag⁺ electrode (RE-7 reference electrode for nonaqueous solvent, produced by BAS Inc.) was used as a reference electrode. Note that the measurements were conducted at room temperature.

The oxidation characteristics of CO11III were examined by 100 cycles of measurements in which a scan for changing the potential of the working electrode with respect to the reference electrode from 0.268 V to 1.05 V and then from 1.05 V to 0.268 V was set to one cycle. Note that the scan rate for the CV measurements was set to 0.1 V/s.

The reduction characteristics of CO11III were examined by 100 cycles of measurements in which a scan for changing the potential of the working electrode with respect to the reference electrode from −1.43 V to −2.45 V and then from −2.45 V to −1.43 V was set to one cycle. Note that the scan rate for the CV measurements was set to 0.1 V/s.

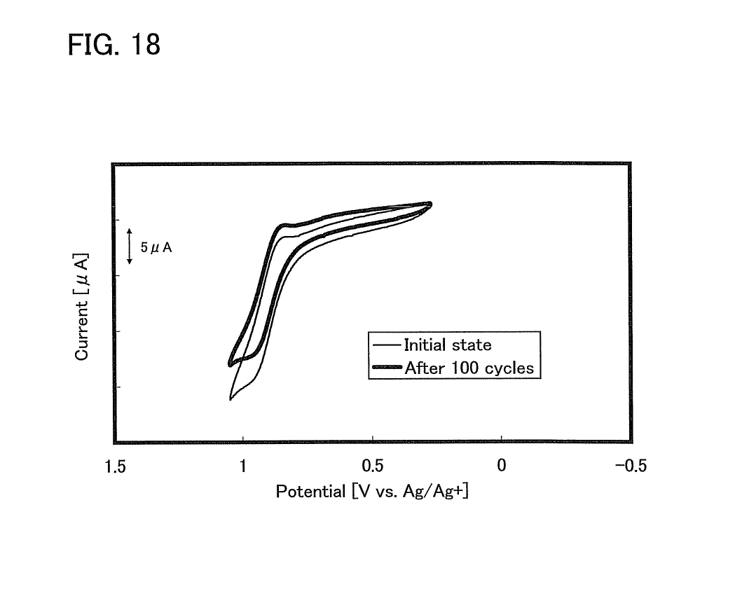

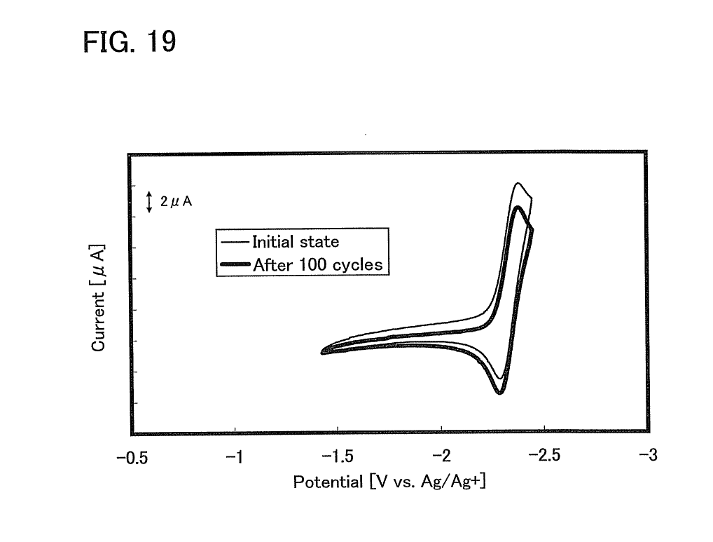

FIG. 18 shows CV measurement results of the oxidation characteristics of CO11III, and FIG. 19 shows CV measurement results of the reduction characteristics of CO11III. In FIG. 18 and FIG. 19, the horizontal axis represents potential (V) of the working electrode with respect to the reference electrode, and the vertical axis represents value of a current (μA) flowing between the working electrode and the auxiliary electrode. As shown in FIG. 18, a current exhibiting oxidation is observed at about +0.91 V (vs. the Ag/Ag⁺ electrode). As shown in FIG. 19, a current exhibiting reduction is observed at about −2.34 V (vs. the Ag/Ag⁺ electrode).

After the 100 cycles of measurements in which the scan was repeated, there was no significant change in the peak position of the CV curves exhibiting the oxidation and reduction reactions. Accordingly, it is found that the oxadiazole derivative that is one embodiment of the disclosed invention is stable to repetitive oxidation-reduction reactions.

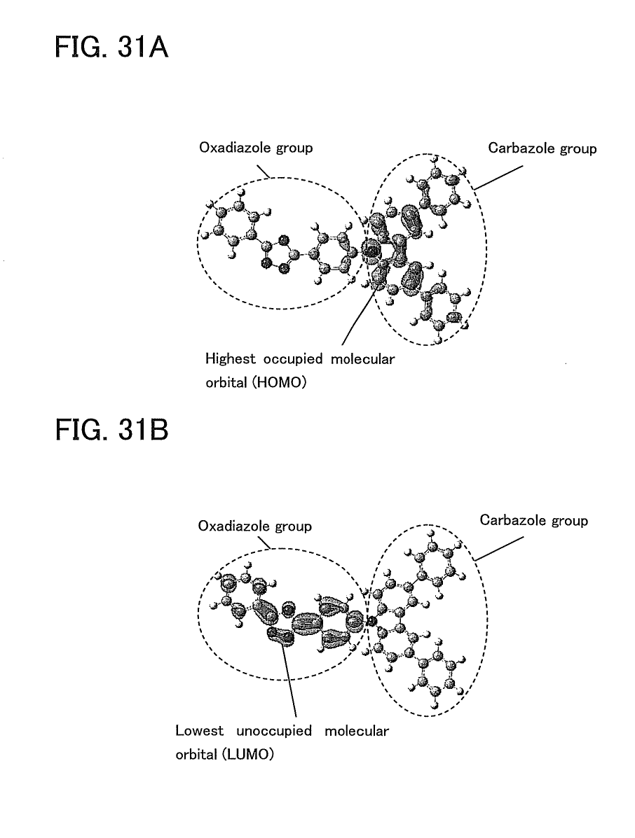

In addition, the optimal molecular structure of CO11III in the ground state was calculated in a manner similar to that of CO11II in the above example. FIGS. 31A and 31B show respectively the highest occupied molecular orbital (HOMO) and lowest unoccupied molecular orbital (LUMO) of CO11III, which were found by the calculations. FIG. 31A shows the highest occupied molecular orbital (HOMO), and FIG. 31B shows the lowest unoccupied molecular orbital (LUMO). In the drawings, the spheres in the drawings represent atoms forming CO11III and cloud-like objects around atoms represent orbits.

FIGS. 31A and 31B demonstrate that the unoccupied molecular orbital and lowest unoccupied molecular orbital of CO11III exist in a carbazole group and an oxadiazole group, respectively. In other words, the carbazole group contributes to the hole-transport property of CO11III while the oxadiazole group contributes to the electron-transport property of CO11III, which indicates the high bipolar character of CO11III.

### Example 3

In this example, description is provided of a method for forming a light-emitting element including any of the oxadiazole derivatives described in the above embodiment as a host material in a light-emitting layer and of results of the element characteristics measurements. Specifically, Light-Emitting Element 1 formed using 3-phenyl-9-[4-(5-phenyl-1,3,4-oxadiazol-2-yl)phenyl]-9H-carbazole (abbreviation: CO11II), which is described in Example 1, and Light-Emitting Element 2 formed using 3,6-diphenyl-9-[4-(5-phenyl-1,3,4-oxadiazol-2-yl)phenyl]-9H-carbazole (abbreviation: COME), which is described in Example 2, will be described.

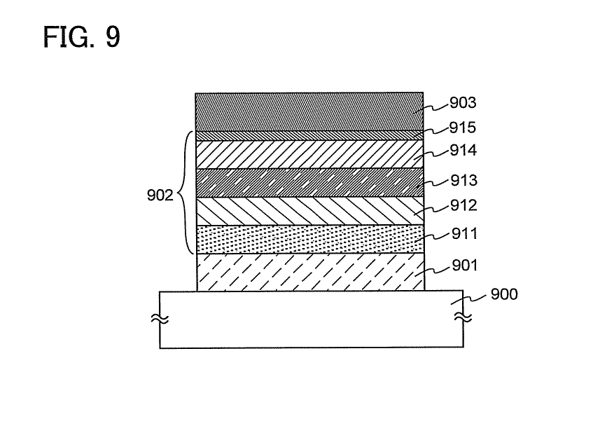

Note that FIG. 9 illustrates a structure of each light-emitting element of this example, in which a third layer 913 which is a light-emitting layer is fanned using one of the above-described oxadiazole derivatives. Structural Formulae of organic compounds used in this example are illustrated below.

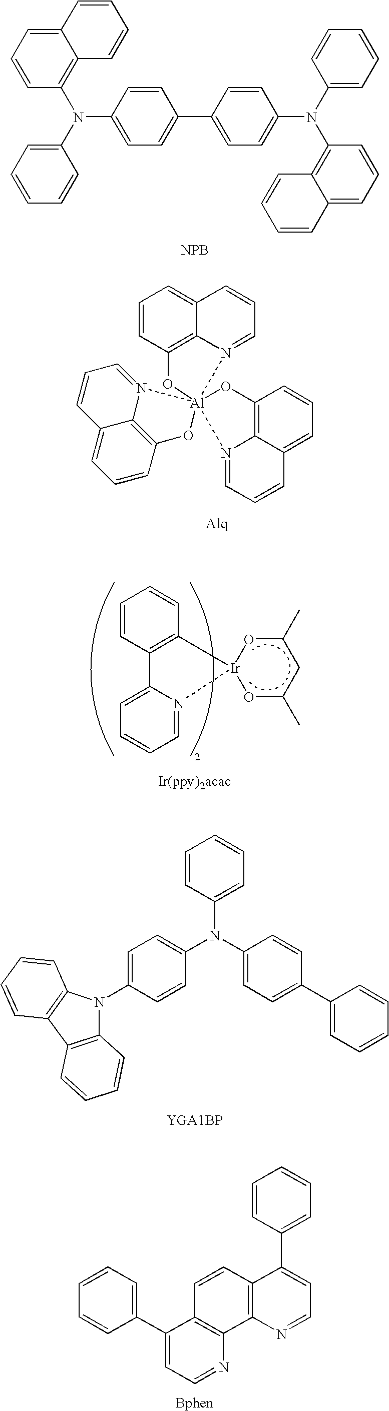

First, indium oxide-tin oxide containing silicon oxide was deposited on a substrate 900 which was a glass substrate by a sputtering method to form a first electrode 901. Note that the thickness of the first electrode 901 was set to 110 nm and the area of the electrode was set to 2 mm×2 mm.

Next, an EL layer 902 including a stack of a plurality of layers was formed over the first electrode 901. In this example, the EL layer 902 has a structure in which a first layer 911 which is a hole-injection layer, a second layer 912 which is a hole-transport layer, the third layer 913 which is a light-emitting layer, a fourth layer 914 which is an electron-transport layer, and a fifth layer 915 which is an electron-injection layer are stacked in that order.

The substrate 900 provided with the first electrode 901 was fixed on a substrate holder that was provided in a vacuum evaporation apparatus so that a surface on which the first electrode 901 was formed faced downward. The pressure in the vacuum evaporation apparatus was reduced to about 10⁻⁴ Pa. Then, on the first electrode 901, 4,4′-bis[N-(1-naphthyl)-N-phenylamino]biphenyl (abbreviation: NPB) and molybdenum(VI) oxide were co-evaporated to form the first layer 911 which was a hole-injection layer. The thickness of the first layer 911 was set to 40 nm, and the evaporation rate was controlled so that the weight ratio of NPB to molybdenum(VI) oxide was 4:1 (=NPB:molybdenum oxide). Note that the co-evaporation method refers to an evaporation method by which evaporation is performed from a plurality of evaporation sources in one treatment chamber simultaneously.

Next, a 20-nm-thick film of a hole-transport material was formed on the first layer 911 by an evaporation method with resistance heating to form the second layer 912 which was a hole-transport layer. Note that for the second layer 912, 4-(9H-carbazol-9-yl)-4′-phenyltriphenylamine (abbreviation: YGA1BP) was used.

Next, the third layer 913 which was a light-emitting layer was formed on the second layer 912 by an evaporation method with resistance heating. As the third layer 913 of Light-Emitting Element 1, 3-phenyl-9-[4-(5-phenyl-1,3,4-oxadiazol-2-yl)phenyl]-9H-carbazole (abbreviation: CO11II) and bis(2-phenylpyridinato-N,C^{2′})iridium(III) acetylacetonato (abbreviation: Ir(ppy)₂acac) were co-evaporated to form a 40-nm-thick film. Here, the evaporation rate was controlled so that the weight ratio of CO11II to Ir(ppy)₂acac was 1:0.06 (=CO11II:Ir(ppy)₂acac). Further, as the third layer 913 of Light-Emitting Element 2, 3,6-diphenyl-9-[4-(5-phenyl-1,3,4-oxadiazol-2-yl)phenyl]-9H-carbazole (abbreviation: CO11III) was used instead of CO11II, and the other conditions were set to the same as those for Light-emitting Element 1.

Furthermore, on the third layer 913, a 10-nm-thick film of tris(8-quinolinolato)aluminum(III) (abbreviation: Alq) and, thereon, a 20-nm-thick film of bathophenanthroline (abbreviation: BPhen) were formed by an evaporation method with resistive heating to form the fourth layer 914 which was an electron-transport layer.

On the fourth layer 914, a 1-nm-thick film of lithium fluoride (LiF) was formed as the fifth layer 915 which was an electron-injection layer.

Lastly, a 200-nm-thick film of aluminum was formed by an evaporation method with resistance heating to form the second electrode 903.

The thus obtained Light-Emitting Elements 1 and 2 were sealed in a glove box containing a nitrogen atmosphere so as not to be exposed to air. Then, operation characteristics of these light-emitting elements were measured. Note that the measurements were carried out at room temperature (25° C.).

Note that for comparison, Light-emitting Element 0 was formed under the same conditions as those for Light-emitting Elements 1 and 2 except for the use of 9-[4-(5-phenyl-1,3,4-oxadiazol-2-yl)phenyl]-9H-carbazole (abbreviation: CO11) as a host material in a light-emitting layer. Note that 9-[4-(5-phenyl-1,3,4-oxadiazol-2-yl)phenyl]-9H-carbazole (abbreviation: CO11) is represented by Structural Formula (300).

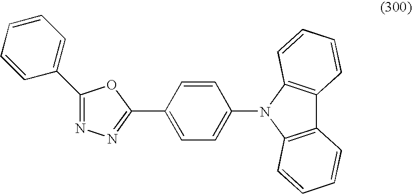

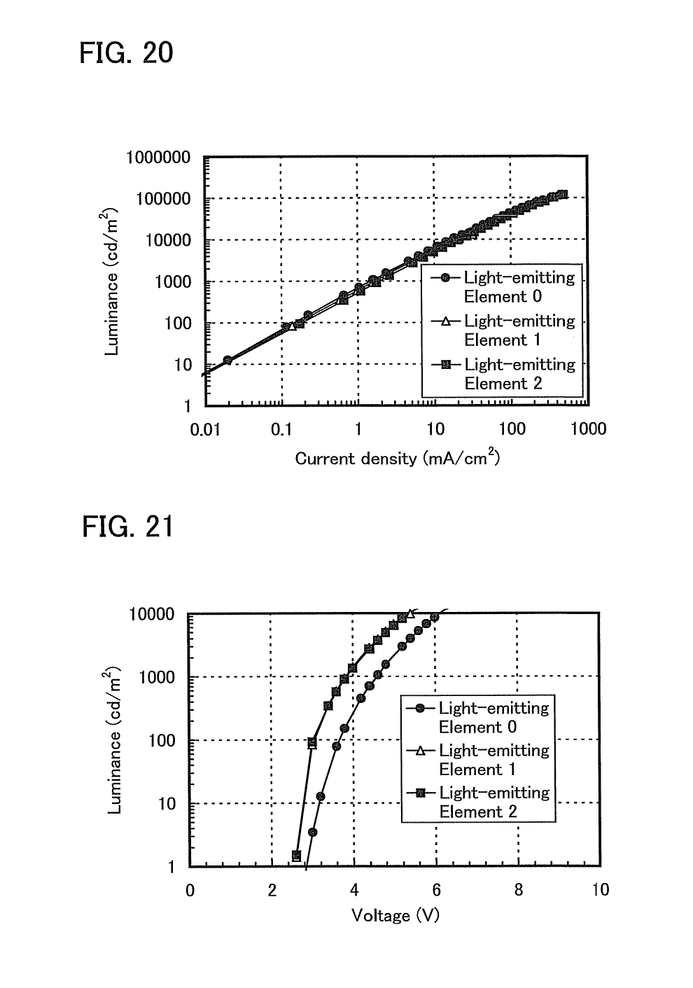

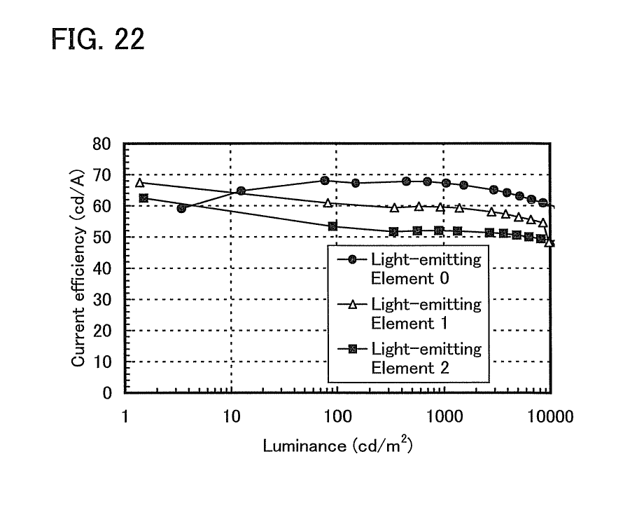

FIG. 20 shows current density vs. luminance characteristics, FIG. 21 shows voltage vs. luminance characteristics, and FIG. 22 shows luminance vs. current efficiency characteristics of Light-emitting Elements 0 to 2. In FIG. 20, the vertical axis represents luminance (cd/m²) and the horizontal axis represents current density (cd/m²). In FIG. 21, the vertical axis represents luminance (cd/m²) and the horizontal axis represents voltage (V). In FIG. 22, the vertical axis represents current efficiency (cd/m²) and the horizontal axis represents luminance (cd/m²). Further, the power efficiency of Light-emitting Element 0 was 46 (lm/W), that of Light-emitting Element 1 was 49 (lm/W), and that of Light-emitting Element 2 was 43 (lm/W). The result is that there is no noticeable difference between these elements in terms of power efficiency.

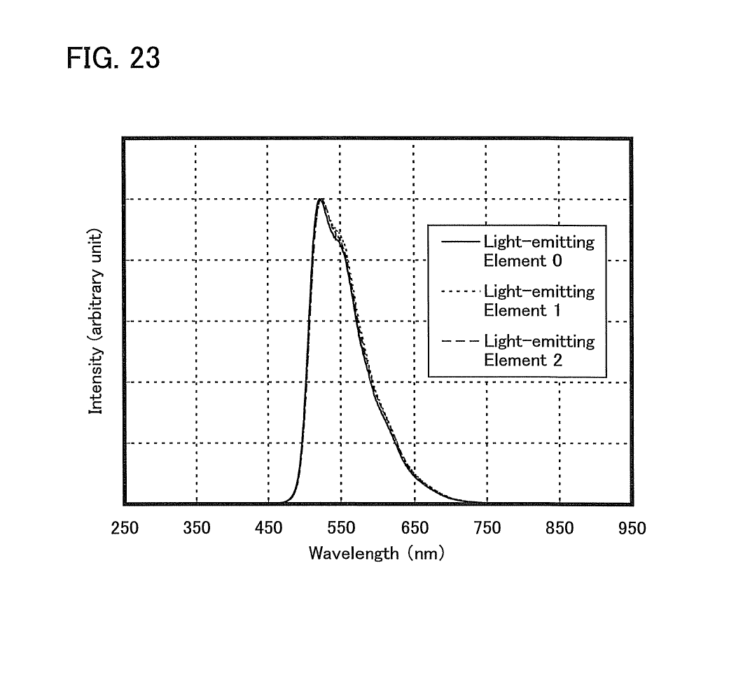

Further, FIG. 23 shows emission spectra obtained by DC constant current driving of Light-emitting Elements 0 to 2 with an initial luminance of 1000 cd/m². As apparent from FIG. 23, the emission spectra of Light-emitting Elements 1 and 2 do not greatly differ from the emission spectrum of Light-emitting Element 0 with which they are compared. Therefore, in both Light-emitting Elements 1 and 2 of this example, the oxadiazole derivatives can be said to serve as the host material.

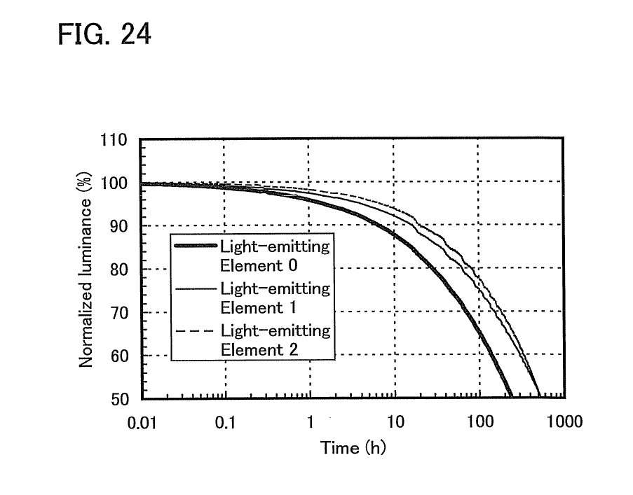

In addition, FIG. 24 shows time vs. normalized luminance characteristics of Light-emitting Elements 0 to 2. In FIG. 24, the vertical axis represents normalized luminance (%), and the horizontal axis represents time (h). As apparent from FIG. 24, Light-emitting Element 1 and Light-emitting Element 2 have respectively a lifetime of about 540 hours and a lifetime of about 530 hours, which shows an improvement in lifetime of each element, while Light-emitting element 0 has a lifetime of about 240 hours. Here, the term lifetime means the length of time the normalized luminance takes to decrease to 50% of the initial luminance.

As described above, Light-emitting Elements 1 and 2 each have even more than twice as long lifetime as Light-emitting Element 0, with which Elements 1 and 2 were compared, despite no noticeable difference in power efficiency. Therefore, use of the oxadiazole derivatives described in the above embodiment as a host material in a light-emitting layer provides a highly reliable light-emitting element having significantly improved lifetime while keeping power consumption.

### Example 4

In this example, description is provided of a method for forming a light-emitting element including any of the oxadiazole derivatives described in the above embodiment as a host material in a light-emitting layer and of results of the element characteristics measurements. Specifically, Light-Emitting Element 3 formed using 3-phenyl-9-[4-(5-phenyl-1,3,4-oxadiazol-2-yl)phenyl]-9H-carbazole (abbreviation: CO11II), which is described in the above example, will be described

Note that a structure of the light-emitting element of this example is illustrated in FIG. 9, in which the third layer 913 which is a light-emitting layer is formed using one of the above-described oxadiazole derivatives.

First, indium oxide-tin oxide containing silicon oxide was deposited on the substrate 900 which was a glass substrate by a sputtering method to form the first electrode 901. Note that the thickness of the first electrode 901 was set to 110 nm and the area of the electrode was set to 2 mm×2 mm.

Next, the EL layer 902 including a stack of a plurality of layers was formed over the first electrode 901. In this example, the EL layer 902 has a structure in which the first layer 911 which is a hole-injection layer, the second layer 912 which is a hole-transport layer, the third layer 913 which is a light-emitting layer, the fourth layer 914 which is an electron-transport layer, and the fifth layer 915 which is an electron-injection layer are stacked in that order.

The substrate 900 provided with the first electrode 901 was fixed on a substrate holder that was provided in a vacuum evaporation apparatus so that a surface on which the first electrode 901 was formed faced downward. The pressure in the vacuum evaporation apparatus was reduced to about 10⁻⁴ Pa. Then, on the first electrode 901, 4-phenyl-4′-(9-phenyl-9H-carbazol-3-yl)triphenylamine (abbreviation: PCBA1BP) and molybdenum(VI) oxide were co-evaporated to form the first layer 911 which was a hole-injection layer. The thickness of the first layer 911 was set to 50 nm, and the evaporation rate was controlled so that the weight ratio of PCBA1BP to molybdenum(VI) oxide was 4:2 (═PCBA1BP:molybdenum oxide). Note that the co-evaporation method refers to an evaporation method by which evaporation is performed from a plurality of evaporation sources in one treatment chamber simultaneously.

Next, a 10-nm-thick film of a hole-transport material was formed on the first layer 911 by an evaporation method with resistance heating to form the second layer 912 which was a hole-transport layer. Note that for the second layer 912, 4-phenyl-4′-(9-phenyl-9H-carbazol-3-yl)triphenylamine (abbreviation: PCBA1BP), which was described above, was used.

Next, the third layer 913 which was a light-emitting layer was formed on the second layer 912 by an evaporation method with resistance heating. In this example, as the third layer 913, 3-phenyl-9-[4-(5-phenyl-1,3,4-oxadiazol-2-yl)phenyl]-9H-carbazole (abbreviation: CO11II), 4-phenyl-4′-(9-phenyl-9H-carbazol-3-yl)triphenylamine (abbreviation: PCBA1BP), and bis{2-(4-fluorophenyl)-3,5-dimethylpyridinato}(picolinate)iridium(III) (abbreviation: Ir(dmFppr)₂pic) were co-evaporated to form a 40-nm-thick film. Here, the evaporation rate was controlled so that the weight ratio of CO11II to PCBA1BP and Ir(dmFppr)₂pic was 1:0.1:0.12 (═CO11II:PCBA1BP:Ir(dmFppr)₂pic). Note that 4-phenyl-4′-(9-phenyl-9H-carbazol-3-yl)triphenylamine (abbreviation: PCBA1BP) is represented by Structural Formula (301) and bis{2-(4-fluorophenyl)-3,5-dimethylpyridinato}(picolinate)iridium(III) (abbreviation: Ir(dmFppr)₂pic) is represented by Structural Formula (302).

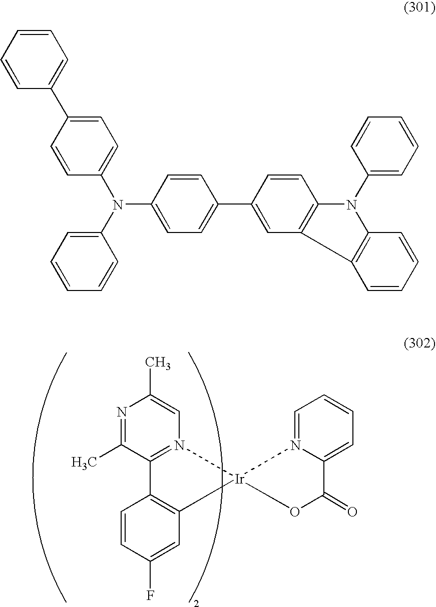

Furthermore, on the third layer 913, a 10-nm-thick film of tris(8-quinolinolato)aluminum(III) (abbreviation: Alq) and, thereon, a 20-nm-thick film of bathophenanthroline (abbreviation: BPhen) were formed by an evaporation method with resistive heating to form the fourth layer 914 which was an electron-transport layer.

On the fourth layer 914, a 1-nm-thick film of lithium fluoride (LiF) was formed as the fifth layer 915 which was an electron-injection layer.

Lastly, a 200-nm-thick film of aluminum was formed by an evaporation method with resistance heating to form the second electrode 903.

The thus obtained Light-Emitting Element 3 was sealed in a glove box containing a nitrogen atmosphere so as not to be exposed to air. Then, operation characteristics of this light-emitting element were measured. Note that the measurements were carried out at room temperature (25° C.).

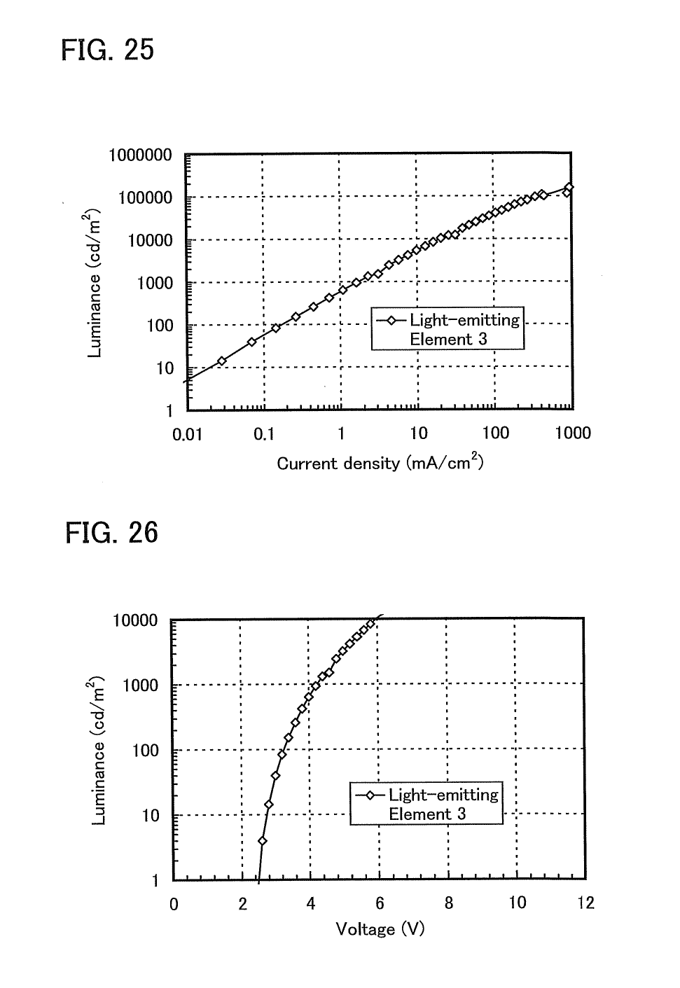

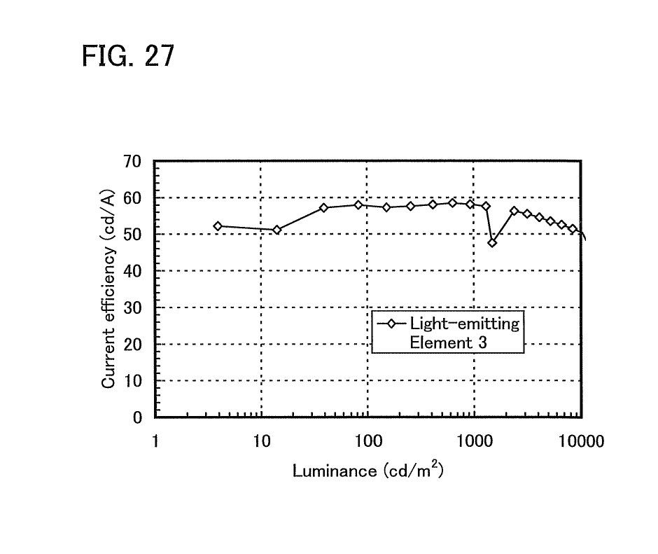

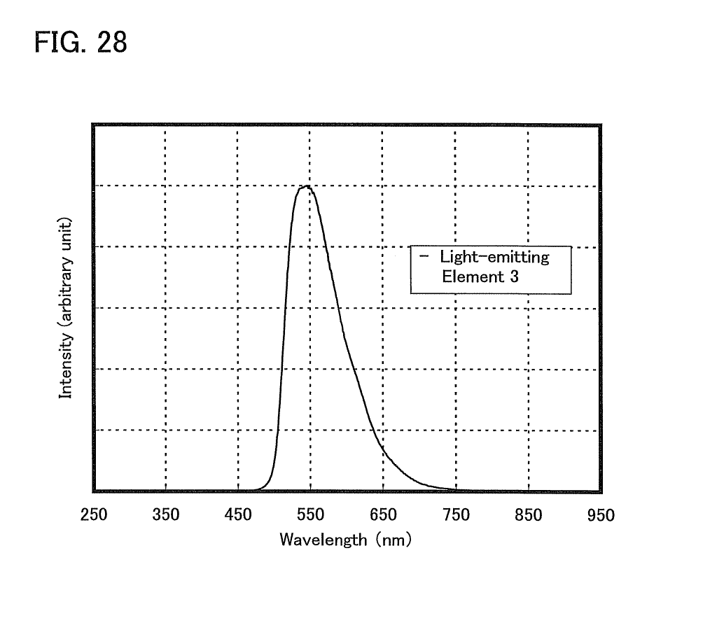

FIG. 25 shows current density vs. luminance characteristics, FIG. 26 shows voltage vs. luminance characteristics, FIG. 27 shows luminance vs. current efficiency characteristics, and FIG. 28 shows an emission spectrum of Light-emitting Element 3. In FIG. 25, the vertical axis represents luminance (cd/m²) and the horizontal axis represents current density (cd/m²). In FIG. 26, the vertical axis represents luminance (cd/m²) and the horizontal axis represents voltage (V). In FIG. 27, the vertical axis represents current efficiency (cd/m²) and the horizontal axis represents luminance (cd/m²). Further, the power efficiency of Light-emitting Element 3 was 44 (lm/W).

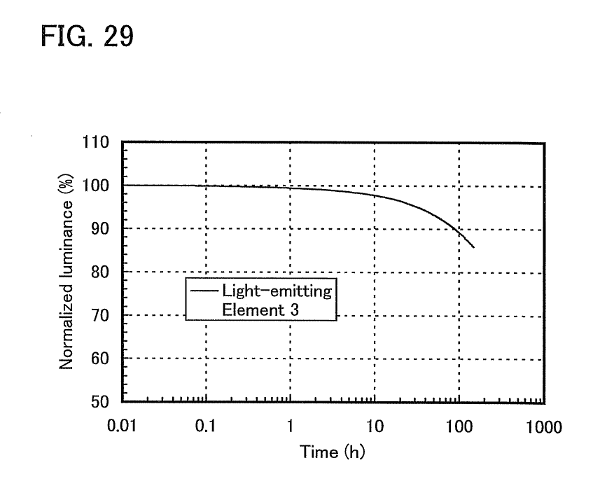

In addition, FIG. 29 shows time vs. normalized luminance characteristics obtained by DC constant current driving of Light-emitting Element 3 with an initial luminance of 1000 cd/m². In FIG. 29, the vertical axis represents normalized luminance (%), and the horizontal axis represents time (h). As apparent from FIG. 29, about 90% of the initial luminance was obtained after 100 hours. The lifetime of Light-emitting Element 3 is assumed to exceed 1000 hours. Here, the term lifetime means the length of time the normalized luminance takes to decrease to 50% of the initial luminance.

As described above, Light-emitting Element 3 has significantly improved lifetime while keeping power efficiency. Therefore, using the oxadiazole derivative described in the above embodiment as a host material in a light-emitting layer provides a highly reliable light-emitting element having significantly improved lifetime while keeping power consumption.

### Reference Example

A method for synthesizing 4-phenyl-4′-(9-phenyl-9H-carbazol-3-yl)triphenylamine (abbreviation: PCBA1BP), which is represented by Structural Formula (301), will be specifically described.

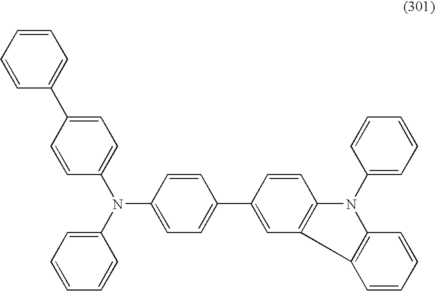

The synthetic scheme of 4-phenyl-4′-(9-phenyl-9H-carbazol-3-yl)triphenylamine (abbreviation: PCBA1BP) is shown in (D-4).

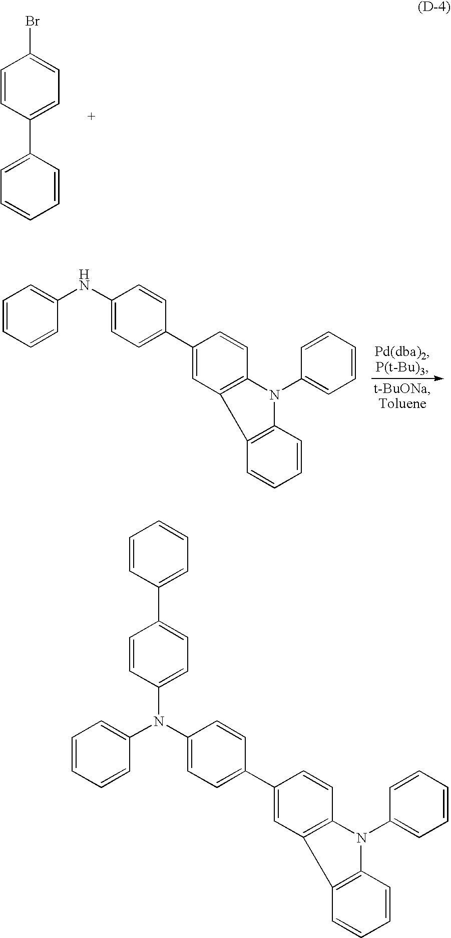

In a 100 mL three-necked flask were placed 2.0 g (4.9 mmol) of 4-(9-phenyl-9H-carbazol-3-yl)diphenylamine, 1.1 g (4.9 mmol) of 4-bromobiphenyl, and 2.0 g (20 mmol) of sodium tert-butoxide, and the atmosphere in the flask was replaced with nitrogen. To this mixture were added 50 mL of toluene and 0.30 mL of tri(tert-butyl)phosphine (10 wt % hexane solution).

This mixture was degassed while being stirred under reduced pressure. After that, 0.10 g of bis(dibenzylideneacetone)palladium(0) was added to this mixture. Next, this mixture was stirred at 80° C. for 5 hours while being heated and reacted. After the reaction, toluene was added to the reaction mixture. The resulting suspension was suction filtered through Celite (Wako Pure Chemical Industries. Ltd., Catalog No. 531-16855), alumina, and Florisil (produced by Wako Pure Chemical Industries, Ltd., Catalog No. 540-00135). The resulting filtrate was washed with an aqueous solution of sodium carbonate and brine in this order. Then, the organic layer was dried by addition of magnesium sulfate. After that, this mixture was suction filtered to remove the magnesium sulfate, whereby a filtrate was obtained.

The obtained filtrate was concentrated, and purification by silica gel column chromatography was performed. The silica gel column chromatography was performed by, first, using a mixed solvent of a 1:9 ratio of toluene to hexane as a developing solvent, and then using a mixed solvent of a 3:7 ratio of toluene to hexane as another developing solvent. The fractions obtained were concentrated to give a solid, which was recrystallized with a mixed solvent of chloroform and hexane to give 2.3 g of a white powdered solid in 84% yield.

By a train sublimation method, 1.2 g of the obtained white solid was purified. Under a reduced pressure of 7.0 Pa with a flow rate of argon at 3 mL/min, the sublimation purification was carried out at 280° C. for 20 hours. The amount of the compound was 1.1 g, and the yield thereof was 89%.

The compound obtained through the above method was measured by a nuclear magnetic resonance ('H NMR) method. The following are the measurement data: ¹H NMR (DMSO-d₆, 300 MHz): δ (ppm)=7.05-7.20 (m, 7H), 7.28-7.78 (m, 21H), 8.34 (d, J=7.8 Hz, 1H), 8.57 (s, 1H)

The measurement results demonstrate that 4-phenyl-4′-(9-phenyl-9H-carbazol-3-yl)triphenylamine (abbreviation: PCBA1BP) represented by Structural Formula (301) was obtained.

By using 4-phenyl-4′-(9-phenyl-9H-carbazol-3-yl)triphenylamine (abbreviation: PCBA1BP), which was described above, Light-emitting Element 3 described in the above example can be formed.

This application is based on Japanese Patent Application serial no. 2009-036626 filed with Japan Patent Office on Feb. 19, 2009, the entire contents of which are hereby incorporated by reference.

What is claimed is:

1. An oxadiazole derivative represented by General Formula (G1), 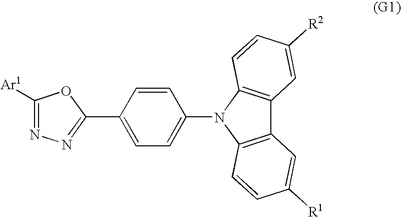
wherein R¹ and R² independently represent a hydrogen atom, an alkyl group having 1 to 4 carbon atoms, or a substituted or unsubstituted aryl group having 6 to 10 carbon atoms, and at least one of R¹ and R² represents a substituted or unsubstituted aryl group having 6 to 10 carbon atoms in a ring, and
wherein Ar¹ represents a substituted or unsubstituted aryl group having 6 to 10 carbon atoms in a ring.

2. An oxadiazole derivative represented by General Formula (G2), 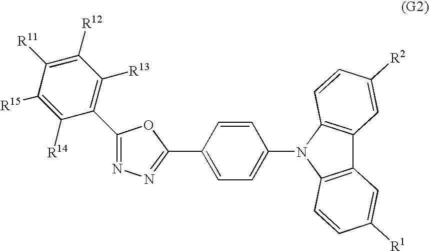
wherein R¹ and R² independently represent a hydrogen atom, an alkyl group having 1 to 4 carbon atoms, or a substituted or unsubstituted aryl group having 6 to 10 carbon atoms, and at least one of R¹ and R² represents a substituted or unsubstituted aryl group having 6 to 10 carbon atoms in a ring, and
wherein R¹¹ to R¹⁵ independently represent a hydrogen atom, an alkyl group having 1 to 4 carbon atoms, or an aryl group having 6 to 10 carbon atoms in a ring.

3. An oxadiazole derivative represented by General Formula (G3), 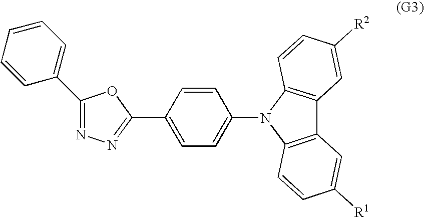
wherein R¹ and R² independently represent a hydrogen atom, an alkyl group having 1 to 4 carbon atoms, or a substituted or unsubstituted aryl group having 6 to 10 carbon atoms, and at least one of R¹ and R² represents a substituted or unsubstituted aryl group having 6 to 10 carbon atoms in a ring.

4. An oxadiazole derivative represented by Structural Formula (G4).

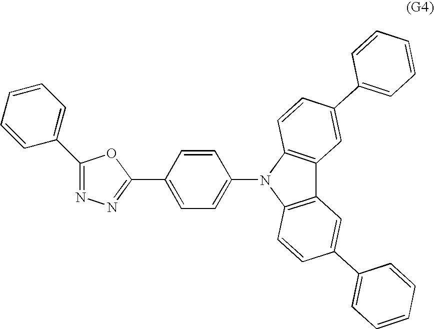

5. An oxadiazole derivative represented by Structural Formula (G5).

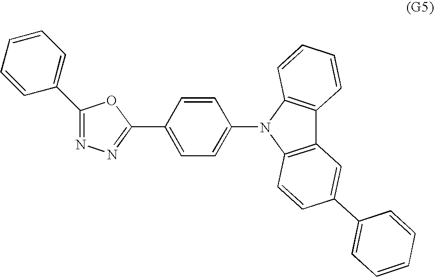

6. A light-emitting element comprising:
a pair of electrodes; and
a layer including an oxadiazole derivative represented by General Formula (G1) between the pair of electrodes, 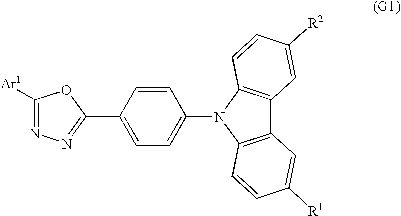
wherein R¹ and R² independently represent a hydrogen atom, an alkyl group having 1 to 4 carbon atoms, or a substituted or unsubstituted aryl group having 6 to 10 carbon atoms, and at least one of R¹ and R² represents a substituted or unsubstituted aryl group having 6 to 10 carbon atoms in a ring, and
wherein Ar¹ represents a substituted or unsubstituted aryl group having 6 to 10 carbon atoms in a ring.

7. The light-emitting element according to claim 6, wherein the layer including the oxadiazole derivative is a light-emitting layer.

8. The light-emitting element according to claim 7, wherein the layer including the oxadiazole derivative further includes a light-emitting substance.

9. The light-emitting element according to claim 8, wherein the light-emitting substance is a phosphorescent compound.

10. The light-emitting element according to claim 6 further comprising a light-emitting layer between the pair of electrodes, wherein the layer comprising the oxadiazole derivative is in contact with the light-emitting layer.

11. A light-emitting element comprising:
a pair of electrodes; and
a layer including an oxadiazole derivative represented by General Formula (G2) between the pair of electrodes, 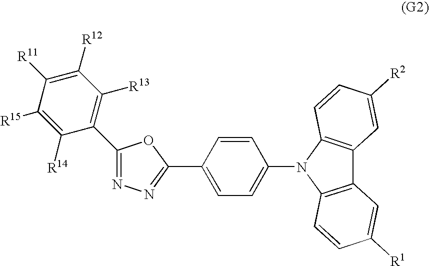
wherein R¹ and R² independently represent a hydrogen atom, an alkyl group having 1 to 4 carbon atoms, or a substituted or unsubstituted aryl group having 6 to 10 carbon atoms, and at least one of R¹ and R² represents a substituted or unsubstituted aryl group having 6 to 10 carbon atoms in a ring, and
wherein R¹¹ to R¹⁵ independently represent a hydrogen atom, an alkyl group having 1 to 4 carbon atoms, or an aryl group having 6 to 10 carbon atoms in a ring.

12. The light-emitting element according to claim 11, wherein the layer including the oxadiazole derivative is a light-emitting layer.

13. The light-emitting element according to claim 12, wherein the layer including the oxadiazole derivative further includes a light-emitting substance.

14. The light-emitting element according to claim 13, wherein the light-emitting substance is a phosphorescent compound.

15. The light-emitting element according to claim 11, further comprising a light-emitting layer between the pair of electrodes, wherein the layer comprising the oxadiazole derivative is in contact with the light-emitting layer.

16. A light-emitting element comprising:
a pair of electrodes; and
a layer including an oxadiazole derivative represented by General Formula (G3) between the pair of electrodes, 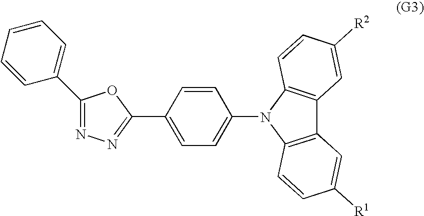
wherein R¹ and R² independently represent a hydrogen atom, an alkyl group having 1 to 4 carbon atoms, or a substituted or unsubstituted aryl group having 6 to 10 carbon atoms, and at least one of R¹ and R² represents a substituted or unsubstituted aryl group having 6 to 10 carbon atoms in a ring.

17. The light-emitting element according to claim 16, wherein the layer including the oxadiazole derivative is a light-emitting layer.

18. The light-emitting element according to claim 17, wherein the layer including the oxadiazole derivative further includes a light-emitting substance.

19. The light-emitting element according to claim 18, wherein the light-emitting substance is a phosphorescent compound.

20. The light-emitting element according to claim 16 further comprising a light-emitting layer between the pair of electrodes, wherein the layer comprising the oxadiazole derivative is in contact with the light-emitting layer.

21. A light-emitting element comprising:
a pair of electrodes; and
a layer including an oxadiazole derivative represented by Structural Formula (G4) between the pair of electrodes.

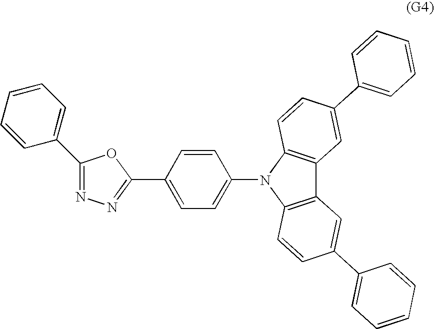

22. The light-emitting element according to claim 21, wherein the layer including the oxadiazole derivative is a light-emitting layer.

23. The light-emitting element according to claim 22, wherein the layer including the oxadiazole derivative further includes a light-emitting substance.

24. The light-emitting element according to claim 23, wherein the light-emitting substance is a phosphorescent compound.

25. The light-emitting element according to claim 21, further comprising a light-emitting layer between the pair of electrodes, wherein the layer comprising the oxadiazole derivative is in contact with the light-emitting layer.

26. A light-emitting element comprising:
a pair of electrodes; and
a layer including an oxadiazole derivative represented by Structural Formula (G5) between the pair of electrodes.

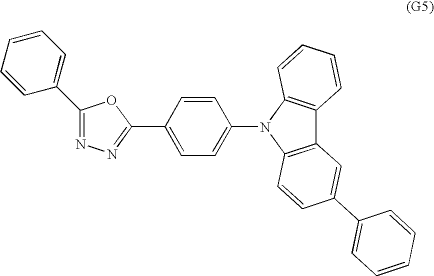

27. The light-emitting element according to claim 26, wherein the layer including the oxadiazole derivative is a light-emitting layer.

28. The light-emitting element according to claim 27, wherein the layer including the oxadiazole derivative further includes a light-emitting substance.

29. The light-emitting element according to claim 28, wherein the light-emitting substance is a phosphorescent compound.

30. The light-emitting element according to claim 26, further comprising a light-emitting layer between the pair of electrodes, wherein the layer comprising the oxadiazole derivative is in contact with the light-emitting layer.
